SEQUENCE LISTING (17 sequences)

SEQ ID NO: 1 - protein, 21 aa, synthetic construct
  VARIANT 7: note = K, Orn, 2,4-diaminobutanoic acid (Dab) or 2,3-diaminopropionic acid (Dap)
  SITE 7: note = Peptide optionally continues from the side chain amino group and not from the alpha-amino group
  VARIANT 18..19: note = KA or AK
AGYLLGXINLKALAALAXXIL

SEQ ID NO: 2 - protein, 19 aa, synthetic construct
  VARIANT 7: note = K, Orn, 2,4-diaminobutanoic acid (Dab) or 2,3-diaminopropionic acid (Dap)
  SITE 7: note = Peptide optionally continues from the side chain amino group and not from the alpha-amino group
  VARIANT 16..17: note = KA or AK
AGYLLGXLKALAALAXXIL

SEQ ID NO: 3 - protein, 21 aa, synthetic construct
  SITE 7: note = Peptide continues from the side chain amino group and not from the alpha-amino group
AGYLLGOINLKALAALAKAIL

SEQ ID NO: 4 - protein, 21 aa, synthetic construct
  SITE 7: note = Peptide continues from the side chain amino group and not from the alpha-amino group
AGYLLGKINLKALAALAKAIL

SEQ ID NO: 5 - protein, 21 aa, synthetic construct
  SITE 7: note = Peptide continues from the side chain amino group and not from the alpha-amino group
  SITE 7: note = 2,4-diaminobutanoic acid (Dab)
AGYLLGXINLKALAALAKAIL

SEQ ID NO: 6 - protein, 21 aa, synthetic construct
  SITE 7: note = 2,4-diaminobutanoic acid (Dab)
AGYLLGXINLKALAALAKAIL

SEQ ID NO: 7 - protein, 21 aa, synthetic construct
  SITE 7: note = Peptide continues from the side chain amino group and not from the alpha-amino group
  SITE 7: note = 2,3-diaminopropionic acid (Dap)
AGYLLGXINLKALAALAKAIL

SEQ ID NO: 8 - protein, 21 aa, synthetic construct
  SITE 7: note = Peptide continues from the side chain amino group and not from the alpha-amino group
  SITE 7: note = Orn
AGYLLGXINLKALAALAAKIL

SEQ ID NO: 9 - protein, 19 aa, synthetic construct
  SITE 7: note = Peptide continues from the side chain amino group and not from the alpha-amino group
  SITE 7: note = Orn
AGYLLGXLKALAALAKAIL

SEQ ID NO: 10 - protein, 19 aa, synthetic construct
  SITE 7: note = Peptide continues from the side chain amino group and not from the alpha-amino group
  SITE 7: note = 2,4-diaminobutanoic acid (Dab)
AGYLLGXLKALAALAKAIL

SEQ ID NO: 11 - protein, 19 aa, synthetic construct
  SITE 7: note = Peptide continues from the side chain amino group and not from the alpha-amino group
  SITE 7: note = 2,4-diaminobutanoic acid (Dab)
AGYLLGXLKALAALAAKIL

SEQ ID NO: 12 - protein, 21 aa, synthetic construct
  SITE 3: note = Y(PO3)
AGYLLGKTNLKALAALAKKIL

SEQ ID NO: 13 - protein, 23 aa, synthetic construct
  SITE 9: note = Peptide continues from the side chain amino group and not from the alpha-amino group
  SITE 9: note = Orn
HHHHYHHGXILLKALKALAKAIL

SEQ ID NO: 14 - protein, 21 aa, synthetic construct
  SITE 7: note = Peptide continues from the side chain amino group and not from the alpha-amino group
  SITE 7: note = Orn
HHYHHGXILLKALKALAKAIL

SEQ ID NO: 15 - protein, 25 aa, synthetic construct
  SITE 11: note = Peptide continues from the side chain amino group and not from the alpha-amino group
  SITE 11: note = Orn
HHHHHHYHHGXILLKALKALAKAIL

SEQ ID NO: 16 - protein, 21 aa, synthetic construct
  SITE 10..11: note = Orn
  SITE 18..19: note = Orn
AGYLLGKLLXXLAAAALXXLL

SEQ ID NO: 17 - protein, 21 aa, synthetic construct
  SITE 10..11: note = Orn
  SITE 14..15: note = Orn
  SITE 18..19: note = Orn
AGYLLGKLLXXLAXXALXXLL

(19) United States
(12) Patent Application Publication
(10) Pub. No.: US 2026/0355330 A1
(43) Pub. Date: Nov. 12, 2026

(54) CELL PENETRATING PEPTIDES AND USES THEREOF

(71) Applicant: VECTIOPEP OÜ, Tartu maakond (EE)

(72) Inventors: Kaido KURRIKOFF, Tartu (EE); Piret ARUKUUSK, Tartu (EE); Ly POROSK, Tartu (EE)

(21) Appl. No.: 19/118,166

(22) PCT Filed: Oct. 4, 2023
(86) PCT No.: PCT/EP2023/077449

§ 371 Date: Apr. 3, 2025

(30) Foreign Application Priority Data
Oct. 4, 2022 (GB) 2214599.9

(51) Int. Cl. A61K 47/64; A61K 39/00; A61K 39/385; A61K 47/54; C07K 7/08

(52) U.S. Cl. CPC A61K 47/6455; A61K 39/385; A61K 47/542; C07K 7/08; A61K 2039/6031

(57) ABSTRACT

The invention relates to novel membrane-permeable constructs and membrane-permeable constructs for use in the transport of cargo across a lipid membrane and subsequent delivery of said cargo into a cell. The cargo may be mRNA and the cell may be in vivo, such as cell of the spleen. Also provided are pharmaceutical compositions comprising the membrane-permeable constructs and use thereof in methods of raising an immune response in a subject.

### FIELD OF THE INVENTION

The invention relates to novel membrane-permeable constructs and membrane-permeable constructs for use in the transport of cargo across a lipid membrane and subsequent delivery of said cargo into a cell. The cargo may be mRNA and the cell may be in vivo, such as cell of the spleen. Also provided are pharmaceutical compositions comprising the membrane-permeable constructs and use thereof in methods of raising an immune response in a subject.

### BACKGROUND

Cell penetrating amino acid sequences (also referred to as cell penetrating peptides or “CPPs”) are relatively short peptides that have the ability to gain access into the cell and mediate the delivery of cargo covalently or non-covalently attached to them.

Cell penetrating amino acids have been used to deliver a multitude of different molecules including, but not limited to, siRNAs. The cell penetrating amino acids sequences typically contain several positively charged amino acids (arginine, lysine, histidine and non-proteogenic amino acid ornithine) and are thus able to associate with the negatively charged backbone of nucleic acid. They form a non-covalent complex mainly via electrostatic interactions, although hydrophobic interactions may play a role in the stability of the formed complexes. The non-covalent cell penetrating amino acid sequence/cargo complexation strategy allows for a simple complex formation with a high mixing versatility, but the limitations of using this approach may include heterogeneity and low stability of the complexes, and the premature or limited dissociation of the cargo from the complexes.

When associated with a cargo, the cell penetrating amino acid sequence/cargo complexes mainly enter via endosomal pathways and end up in the endosomal compartments or in the lysosomes. In order to take its intended effect, the internalisation has to be promptly followed by the release of complexes from these organelles.

The cell membrane has an amphiphilic nature. To enhance cell penetrating amino acid membrane interactions, several cell penetrating amino acid sequences have been designed to have both hydrophilic and hydrophobic regions and/or moieties in their sequence. NickFect55 (NF55) is a cell penetrating peptide, useful as a plasmid DNA (pDNA) delivery vector for nucleic acid delivery applications in vitro and in vivo. It has an N-terminal fatty acid and a non-proteinogenic amino acid, ornithine in the aa7 position. It was initially designed for the delivery of pDNA with an optimal CPP/pDNA complex stability and positive charges distributed along theoretical alpha-helix. Several amphipathic CPPs have previously been used to efficiently deliver siRNAs into the mammalian cells.

mRNA therapeutics are a class of drugs that allow two types of therapeutic approaches: mRNA-based vaccination; and mRNA as protein replacement therapy. The COVID-19 pandemic (caused by the SARS-CoV2 virus) has granted mRNA vaccines unprecedented attention (Dolgin E. (2021) Nature, 589(7841):189-191, doi: https://doi.org/10.1038/d41586-021-00019-w) and although frequently presented as a “new technology” they are actually the result and combination of past technological breakthroughs and discoveries from various disciplines Pardi et al. (2018) Nat Rev Drug Discov, 17(4):261-279, doi: https//doi.org/10.1038/nrd.2017.243). The most critical of those technological components is mRNA delivery technology (Le et al. (2020) Trends Biochem Sci, 46(5):351-365, doi: https://doi.org/10.1016/j.tibs.2020.11.010) and it is currently the bottleneck (Sahin et al. (2014) Nat Rev Drug Discov, 13(10):759-80, doi: https://doi.org/10.1038/nrd4278) that defines the potential of the whole platform. Current mRNA vaccine technology is based on liposomes (LNPs; Corbett et al. (2020) Nature, 586(7830):567-571, doi: https://doi.org/10.1038/s41586-020-2622-0) and impressive development in the past 10 years has advanced these to in vivo systemic use (Semple et al. (2010) Nature Biotechnology, 28(2):172-176, doi: https://doi.org/10.1038/nbt.1602; and Dong et al. (2014) Proc Natl Acad Sci USA, 111(11):3955-60, doi: https://doi.org/10.1073/pnas.1322937111). Nevertheless, the greatest controversy and challenge for the LNPs have been: (i) their side effects; and (ii) the fact that nucleic acid delivery—albeit effective—is restricted to the liver tissue. In order to fully tackle the possibilities of the mRNA therapeutics, the delivery technology has to be improved. For example, the current SARS-CoV2 vaccination regime requires local administration of the LNP and a low dose of mRNA, which is sufficient to activate the immune system (Servick, K. (2020) Science News, https://www.science.org/content/article/mysterious-2-billion-biotech-revealing-secrets-behind-its-new-drugs-and-vaccines). However, in order to treat chronic life-threatening diseases, the delivery technology has to be: (i) significantly more efficient; and (ii) less toxic.

CPPs have previously been developed from the NickFect (NF) and PepFect (PF) families for plasmid (pDNA), siRNA and miRNA delivery. In the PepFect family, PF14 has been used successfully for pDNA (Veimann et al. (2013) Mol. Pharmaceutics, 10(1):199-210, doi: https://doi.org/10.1021/mp3003557) and siRNA delivery (Sirmane et al. (2018) Peptides, 104(6):62-69, doi: https://doi.org/10.1016/j.peptides.2018.04.015), both in vitro and in vivo. The present inventors have also previously shown that PF1452 (Kurrikoff et al. (2017) Sci. Rep., 7(1):17056, doi: https://doi.org/10.1038/s41598-017-17316-y) is even more efficient than PF14 for the delivery of pDNA in vivo. NF55 (Freiman et al. (2016) J of Control. Release, 241(10):135-143, doi: https://doi.org/10.1016/j.jconrel.2016.09.022) is an excellent vector for the delivery of DNA in vivo. In Porosk et al. (2019, Biomater. Sci., 7:4363-4374, doi: https://doi.org/10.1039/c9bm00688e), the present inventors designed and tested a series of histidine containing peptides for siRNA delivery and found that NF70 and NF71 were the most efficient, even more efficient than NF55. In Carreras-Badosa et al. (2020, Biomaterials 262:120316, doi: https://doi.org/10.1016/j.biomaterials.2020.120316), different NF and PF family CPPs (PF14, NF55, NF70 and NF71) were tested for miRNA delivery. Of all the tested peptides, NF71 was the most efficient in vivo. NF70 has also been used to transfect therapeutic siRNA in vitro and in vivo (Kiisholts et al. (2021) Pharmaceutics, 13 (10): 1618, doi: https://doi.org/10.3390/pharmaceutics13101618). Therefore, while several NFs and PFs have been developed for and shown to deliver pDNA, siRNA and miRNA, no studies have to date demonstrated good delivery of mRNA. Furthermore, in van den Brand et al. (2019, E J of Pharmaceutics and Biopharmaceutics, 141:180-190, doi: https://doi.org/10.1016/j.ejpb.2019.05.014), PF14 was used for the delivery of mRNA with limited success.

There is therefore a great need to develop CPPs and membrane-permeable constructs capable of improved delivery of cargoes, in particular the delivery of mRNA in vivo, such as to the spleen. Such CPPs and membrane-permeable constructs may then be used in therapy, e.g. for raising an immune response when used as part of an mRNA vaccine.

### SUMMARY OF THE INVENTION

According to a first aspect of the invention, there is provided a membrane-permeable construct for use in the transport of cargo across a lipid membrane and subsequent delivery of said cargo into a cell in vivo, wherein the construct comprises a cell penetrating amino acid sequence and fatty acid chain attached to the N-terminus of said amino acid sequence, wherein the cargo is mRNA, wherein the cell penetrating amino acid sequence comprises the sequence of:

(SEQ ID NO: 1)
AGYLLG X₁* INLKALAALA X₂ IL;
or
(SEQ ID NO: 2)
AGYLLG X₁* LKALAALA X₂ IL,

wherein X₁ represents Lys (K), Orn (O), Dab or Dap,
wherein * indicates that the peptide optionally continues from the side chain amino group and not from the α-amino group,
wherein X₂ represents KA or AK, and
wherein the cell penetrating amino acid sequence is optionally chemically modified at the C-terminus.

According to a second aspect of the invention, there is provided a membrane-permeable construct for transport of cargo across a lipid membrane and subsequent delivery of said cargo into a cell, wherein the construct comprises a cell penetrating amino acid sequence and fatty acid chain attached to the N-terminus of said amino acid sequence, wherein the cell penetrating amino acid sequence comprises the sequence of:

(SEQ ID NO: 2)
AGYLLG X₁* LKALAALA X₂ IL,

wherein X₁ represents Lys (K), Orn (O), Dab or Dap,
wherein * indicates that the peptide continues from the side chain amino group and not from the α-amino group, and
wherein X₂ represents KA or AK.

In particular embodiments, the cargo is mRNA. In further particular embodiments, the cell is in vivo, and/or wherein the cell is a cell of the spleen, such as a dendritic cell (DC).

In another aspect, there is provided a pharmaceutical composition comprising the membrane-permeable construct described herein and a pharmaceutically acceptable carrier, optionally further comprising one or more adjuvants.

In a yet further aspect, there is provided a membrane-permeable construct for use or the membrane-permeable construct described herein or the pharmaceutical composition described herein for use in a method of raising an immune response in a subject. Such immune response being therapeutically useful or prophylactically useful, such as in a vaccine.

### BRIEF DESCRIPTION OF THE FIGURES

FIG. 1: Delivery of reporter mRNA with CPPs in cell culture—evaluation of reporter levels post-transfection (relative light units normalised to total protein in the cell lysate (RLU/mg)). Naked mRNA treated cells were used as controls and Lipofectamine3000 (LF3000) was used to compare the transfection efficacy with a liposome-based transfection reagent.

FIG. 2: Comparison of reporter levels in cells post-transfection of pDNA or mRNA delivered by CPPs (P14, NF55 and NF71; relative light units normalised to total protein in the cell lysate (RLU/mg)). Naked mRNA and untreated cells (UT) were used as controls.

FIG. 3: Biodistribution of reporter levels post-transfection of mRNA or pDNA with NFs or PFs CPPs in vivo. Reporter gene product levels were evaluated from the whole tissue homogenates collected post mortem. Results are shown as the fold change to mRNA or pDNA treated mice.

FIG. 4: Live animal imaging 1-48 hours post administration of NF424/mRNA or NF424/pDNA complexes. Reporter gene product levels were evaluated with full body imager at 1 h, 6 h, 12 h, 24 h, and 48 h post-injection.

FIG. 5: Delivery of mRNA with CPPs in vivo—reporter levels in the spleen. Spleens were harvested post mortem 16 hours post-injection. The reporter levels (RLU) were evaluated from the whole tissue homogenate.

FIG. 6: Reporter levels detected from spleen cells subtypes collected from NF55/mRNA treated mice. Reporter protein levels were evaluated from the sorted splenocytes: T cells, Macrophages, Dendritic cells and the rest of the lymphocytes. Results are shown as the fold change compared to untreated mice.

### BRIEF DESCRIPTION OF THE SEQUENCES

SEQ ID NO: 1 represents a consensus amino acid sequence of the cell penetrating amino acid sequence comprised in the membrane-permeable constructs described herein for use in the transport of cargo across a lipid membrane and subsequent delivery of said cargo into a cell in vivo, wherein the cargo is mRNA.

SEQ ID NO: 2 represents a consensus amino acid sequence of the cell penetrating amino acid sequence comprised in the novel membrane-permeable constructs described herein.

SEQ ID NO: 3 represents an amino acid sequence comprised in the membrane-permeable constructs of the first aspect of the invention, NF419, NF55, NF410 and NF411.

SEQ ID NO: 4 represents an amino acid sequence comprised in the membrane-permeable constructs of the first aspect of the invention, NF420, NF554, NF412 and NF413.

SEQ ID NO: 5 represents an amino acid sequence comprised in the membrane-permeable constructs of the first aspect of the invention, NF422, NF553 and NF423.

SEQ ID NO: 6 represents an amino acid sequence comprised in the membrane-permeable construct of the first aspect of the invention, NF559.

SEQ ID NO: 7 represents an amino acid sequence comprised in the membrane-permeable construct of the first aspect of the invention, NF550.

SEQ ID NO: 8 represents an amino acid sequence comprised in the membrane-permeable construct of the first aspect of the invention, NF54.

SEQ ID NO: 9 represents an amino acid sequence comprised in the membrane-permeable constructs of the second aspect of the invention, NF430 and NF437.

SEQ ID NO: 10 represents an amino acid sequence comprised in the membrane-permeable constructs of the second aspect of the invention, NF425, NF424 and NF426.

SEQ ID NO: 11 represents an amino acid sequence comprised in the membrane-permeable constructs of the second aspect of the invention, NF436 and NF438.

SEQ ID NOs: 12 to 17 represent amino acid sequences comprised in control/comparator membrane-permeable constructs utilised herein (NF1, NF70, NF71, NF72, PF14, PF1452).

### DETAILED DESCRIPTION OF THE INVENTION

According to a first aspect of the invention, there is provided a membrane-permeable construct for use in the transport of cargo across a lipid membrane and subsequent delivery of said cargo into a cell in vivo, wherein the construct comprises a cell penetrating amino acid sequence and fatty acid chain attached to the N-terminus of said amino acid sequence, wherein the cargo is mRNA, wherein the cell penetrating amino acid sequence comprises the sequence of:

(SEQ ID NO: 1)
AGYLLG X₁* INLKALAALA X₂ IL;
or
(SEQ ID NO: 2)
AGYLLG X₁* LKALAALA X₂ IL,

wherein X₁ represents Lys (K), Orn (O), Dab or Dap,
wherein * indicates that the peptide optionally continues from the side chain amino group and not from the α-amino group,
wherein X₂ represents KA or AK, and
wherein the cell penetrating amino acid sequence is optionally chemically modified at the C-terminus.

The inventors have found that membrane-permeable constructs according to this first aspect of the present invention have surprisingly good ability to deliver mRNA cargo into the intracellular compartment, in particular into cells of the spleen in vivo. Such delivery is predicted to be efficient for raising an immune response in a subject, such as a priming or vaccination response, since professional antigen presenting cells (APCs), such as dendritic cells (DCs), relocate to secondary lymphoid organs like the spleen following exposure to antigen and the spleen contains the largest number of APCs (including DCs) in mammals.

Therefore, all membrane-permeable constructs and their uses as described herein are likely to be greatly improved compared to commonly used liposome (LNP) delivery systems which target cargo to liver tissue and comprise polyethylene glycol (PEG), and thus have side effects and toxicity after systemic administration (Semple et al. (2010); and Dong et al. (2014)). By effectively delivering cargo (e.g. mRNA) to the spleen, the present invention yields higher efficiency and lower toxicity than liposomes/LNPs. Furthermore, because of their reduced toxicity, all of the membrane-permeable constructs described herein may be used to deliver higher doses of cargo, in particular mRNA, than liposomes/LNPs which to date have used a low dose due to their toxicity, side effects and liver-restricted delivery. As such, the membrane-permeable constructs described herein may also find utility in methods of treating chronic diseases.

In addition, all of the membrane-permeable constructs of the present invention are easier to formulate with nucleic acids (e.g. mRNA) than liposomes/LNPs because of their ability to spontaneously complex (e.g. form a nanoparticle). This is in part due to only one amino acid component being present compared to the four components that make up a liposome/LNP capsule, and also because of the neutralisation of the nucleic acid (e.g. mRNA) by the peptide backbone due to the presence of positively charged amino acids, such as ornithine (Orn, O) and lysine (K). All membrane-permeable constructs described herein also provide a completely biodegradable delivery platform, with no accumulation or adverse effects in cells even long after delivery.

Thus, in certain embodiments all membrane-permeable constructs described herein find particular utility in delivering nucleic acid constructs which are large in size and linear, such as messenger RNA (mRNA), and which encode an antigenic protein or peptide. It is therefore expected that the membrane-permeable constructs described herein may be effectively used for delivering mRNA encoding antigenic proteins or peptides in methods of raising an immune response to the antigenic proteins/peptides in a subject, or may be administered to a subject as part of a method for raising such an immune response in said subject.

### Construct

“Constructs” as referred to herein comprise an amino acid peptide sequence of any length with a fatty acid chain attached to the N-terminus of said amino acid sequence. The constructs described herein may also further comprise a cargo, in particular an mRNA cargo.

The construct is membrane-permeable. Thus, it may pass from the extracellular environment across or through a membrane (e.g. a lipid membrane) into a cell or intracellular environment. Membranes may be single or multiple layer structures (e.g. a lipid bilayer). The construct may transport the cargo across a membrane and deliver the cargo into the cytoplasm of the cell. The construct may also transport the cargo across a membrane and deliver the cargo to an organelle in the cell. Alternatively or additionally, the construct may transport cargo across a membrane and deliver the cargo to an intracellular surface. The construct may enter the cell via an endosomal pathway. The construct may be encapsulated in an intra-organelle, preferably in an endosomal compartment or in a lysosome, and subsequently be released into the intracellular compartment or environment.

The membrane may be an artificial membrane such as an artificially constructed complex membrane formed of, for example, lipids, phospholipids or molecules having both hydrophilic and hydrophobic compounds or structures. The membrane may be a biological membrane, including but not limited to eukaryotic cell membranes and prokaryotic cell membranes. The membrane may be a lipid bilayer or phospholipid bilayer. The membrane may be a lipid membrane of a phospholipid membrane. The membrane may be a plasma membrane, such as the plasma membrane of a cell. Thus, in one embodiment the membrane-permeable constructs disclosed herein are for transport of cargo across a lipid membrane and subsequent delivery of said cargo to a cell. In another embodiment, the membrane-permeable constructs disclosed herein are for use in such transport. Eukaryotic cell membranes include, but are not limited to, membranes of: immune cells, such as white blood cells, red blood cells, monocytes, macrophages, neutrophils, T cells, B cells or dendritic cells; epithelial cells; endothelial cells; keratinocytes; muscle cells; skin cells; nerve cells and fat cells.

### Cell Penetrating Amino Acid Sequences

Cell penetrating amino acid sequences, also referred to as cell penetrating peptides or “CPPs”, are short amino acid sequences that transport different types of cargo molecules across a lipid membrane and facilitate cellular uptake of the cargo molecules. Cell penetrating amino acid sequences may comprise between 19 to 25 or 25 to 30 amino acids. Cell penetrating amino acid sequences may comprise 19 or more, 20 or more, 21 or more, 22 or more, 23 or more, 24 or more, 25 or more, 26 or more, 27 or more, 28 or more, 29 or more or 30 or more amino acids. Cell penetrating amino acid sequences may comprise 20, 21, 22, 23, 24, 25, 26, 27, 28, 29 or 30 amino acids. Thus, in some embodiments the cell penetrating amino acid sequence comprises 19 or more, 20 or more or 21 or more amino acids. In further embodiments, the cell penetrating amino acid sequence comprises 19, 20 or 21 amino acids.

A property of cell penetrating amino acids described herein is their ability to translocate the lipid membrane and facilitate the delivery of various molecular cargoes to the cytoplasm or to an organelle of a cell or to an intracellular cell surface. In certain embodiments, the cell penetrating amino acids as comprised in a membrane-permeable construct described herein deliver a cargo to the cytoplasm of a cell, such that wherein the cargo is mRNA it is available to the translation machinery (e.g. ribosomes etc.) to generate the protein or peptide encoded by said mRNA, such as an antigenic protein or peptide.

In one embodiment, the cell penetrating amino acid sequence comprises or consists of:

(SEQ ID NO: 1)
AGYLLG X₁* INLKALAALA X₂ IL.

According to this embodiment:
X₁ represents Lys (K), Orn (O), Dab or Dap,
* indicates that the peptide optionally continues from the side chain amino group and not from the α-amino group, and
X₂ represents KA or AK.

Dab refers to 2,4-diaminobutanoic acid. Dap refers to 2,3-diaminopropionic acid.

The natural proteogenic amino acids and the non-proteogenic (also known as non-natural) amino acids of the cell penetrating peptide may be in the naturally occurring L-enantiomer configuration (which may be represented by upper case letters).

In a further embodiment, X₁ is K, Orn (O) or Dab. In a particular embodiment X₁ is Dab. In a yet further embodiment, the peptide continues from the side chain amino group and not from the α-amino group at the * position. In a still further embodiment, X₂ is KA.

In another embodiment, the cell penetrating amino acid sequence comprises or consists of:

(SEQ ID NO: 2)
AGYLLG X₁* LKALAALA X₂ IL.

According to this embodiment:
X₁ represents K, Orn (O), Dab or Dap,
* indicates that the peptide continues from the side chain amino group and not from the α-amino group, and
X₂ represents KA or AK.

Thus, in a second aspect of the invention there is provided a membrane-permeable construct for transport of cargo across a lipid membrane and subsequent delivery of said cargo into a cell, wherein the construct comprises a cell penetrating amino acid sequence and fatty acid chain attached to the N-terminus of said amino acid sequence, wherein the cell penetrating amino acid sequence comprises the sequence of:

(SEQ ID NO: 2)
AGYLLG X₁* LKALAALA X₂ IL,

wherein X₁ represents Lys (K), Orn (O), Dab or Dap,
wherein * indicates that the peptide continues from the side chain amino group and not from the α-amino group, and
wherein X₂ represents KA or AK.

In a further embodiment, X₁ is Orn (O) or Dab. In a particular embodiment X₁ is Dab. In a particular embodiment, X₂ is KA. In another particular embodiment, X₂ is AK.

The inventors have found that the novel membrane-permeable constructs according to this second aspect of the invention have good ability to deliver cargo into an intracellular compartment, such as cargoes selected from: mRNA, plasmid DNA (pDNA), small-interfering RNA (siRNA), micro RNA (miRNA), or any other coding or non-coding nucleic acid, as well as peptides, proteins, non-peptidic pharmaceutical agents, polysaccharides, lipids (including lipoproteins and glycolipids), small molecule drugs and imaging agents. As with membrane-permeable constructs of the first aspect herein, such delivery is predicted to be efficient for raising an immune response in a subject, such as a priming or vaccination response, since professional antigen presenting cells (APCs), such as dendritic cells (DCs), relocate to secondary lymphoid organs like the spleen following exposure to antigen and the spleen contains the largest number of APCs (including DCs) in mammals, in particular when the cargo is mRNA encoding an antigenic protein or peptide. However, as will be appreciated from the disclosures herein the membrane-permeable constructs according to the second aspect of the invention are not limited for use in the transport of mRNA across a lipid membrane and subsequent delivery of said mRNA into a cell, and thus may be for the transport of any suitable cargo.

In some embodiments, the membrane-permeable constructs are more effective at cargo (e.g. mRNA) delivery than existing cell penetrating peptides, for example NF55 or NF54. In particular embodiments, the membrane-permeable constructs according to the second aspect of the invention are more effective at cargo (e.g. mRNA) delivery than existing cell penetrating peptides, for example NF55 or NF54. The constructs also, in at least some embodiments, advantageously have low toxicity and are expected to be cheaper to synthesise compared to existing cell penetrating peptides, for example NF55 or NF54.

Throughout the present disclosure, a residue and/or position number in the cell penetrating amino acid sequence refers to a position in the sequence by reference to the numbering of SEQ ID NO: 1 or SEQ ID NO: 2 as appropriate, it being understood that the said sequences may have any or none of the modifications described herein. Therefore, by way of example only, the numbering of residues/positions within the cell penetrating peptide sequences described herein can be represented as follows:

(SEQ ID NO: 3)
A₁ G₂ Y₃ L₄ L₅ G₆ O₇* I₈ N₉ L₁₀ K₁₁ A₁₂ L₁₃ A₁₄
A₁₅ L₁₆ A₁₇ K₁₈ A₁₉ I₂₀ L₂₁;
or
(SEQ ID NO: 9)
A₁ G₂ Y₃ L₄ L₅ G₆ O₇* L₈ K₉ A₁₀ L₁₁ A₁₂ A₁₃ L₁₄
A₁₅ K₁₆ A₁₇ I₁₈ L₁₉.

Furthermore, the “N-terminal part” referred to herein comprises the amino acid residues at the ‘end’ of the cell penetrating peptide amino acid sequence which is the N-terminus. Thus, in one embodiment the N-terminal part comprises positions 1 to 6 of SEQ ID NO: 1 or SEQ ID NO: 2. In another embodiment, the N-terminal part comprises positions 1 to 5 of SEQ ID NO: 1 or SEQ ID NO: 2. The N-terminal part of the cell penetrating amino acid sequence may also include amino acids at positions 1 to 4, 1 to 3, 1 to 2, 2 to 6, 2 to 5, 2 to 4, 2 to 3, 3 to 6, 3 to 5 and 3 to 4 of the cell penetrating amino acid sequence. Substitution refers to the replacement of an amino acid with another different amino acid.

In some embodiments, the cell penetrating amino acid sequence comprises one or more further amino acid substitution at positions 9 to 21 of SEQ ID NO: 1 or at positions 9 to 19 of SEQ ID NO: 2.

Thus, in some embodiments the cell penetrating amino acid sequence comprises or consists of the sequence of:

(SEQ ID NO: 3)
AGYLLGO*INLKALAALAKAIL;
(SEQ ID NO: 4)
AGYLLGK*INLKALAALAKAIL;
(SEQ ID NO: 5)
AGYLLGDab*INLKALAALAKAIL;
(SEQ ID NO: 6)
AGYLLGDabINLKALAALAKAIL;
(SEQ ID NO: 7)
AGYLLGDap*INLKALAALAKAIL;
or
(SEQ ID NO: 8)
AGYLLGO*INLKALAALAAKIL,

wherein * indicates that the peptide continues from the side chain amino group and not from the α-amino group.

In particular embodiments, the cell penetrating amino acid sequence comprises or consists of the sequence of:

(SEQ ID NO: 3)
AGYLLGO*INLKALAALAKAIL;
(SEQ ID NO: 4)
AGYLLGK*INLKALAALAKAIL;
(SEQ ID NO: 5)
AGYLLGDab*INLKALAALAKAIL;
or
(SEQ ID NO: 8)
AGYLLGO*INLKALAALAAKIL

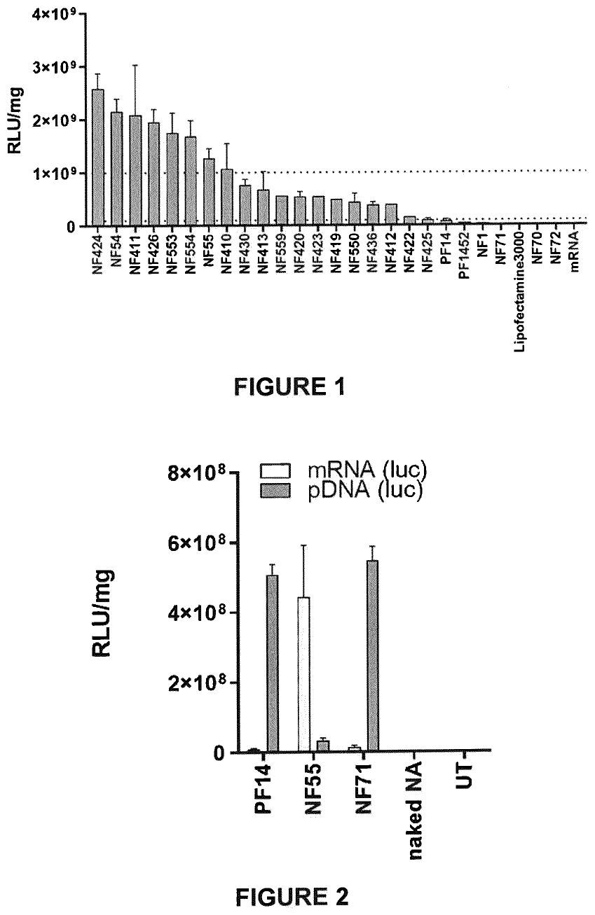

As demonstrated by the data presented herein, membrane-permeable constructs comprising cell penetrating amino acid sequences according to these embodiments provide better or equivalent delivery of an mRNA cargo than the existing cell penetrating peptides, NF55 and NF54. In particular, certain cell penetrating peptides comprising sequences of SEQ ID NO: 3, SEQ ID NO: 4, SEQ ID NO: 5 or SEQ ID NO: 8 provided significantly better delivery of an mRNA cargo, leading to significantly increased activity of the expressed protein (i.e. increased expression of said protein), compared to NF55 (see FIG. 1).

In one embodiment, the membrane-permeable construct for use in the transport of mRNA across a lipid membrane and subsequent delivery of said mRNA into a cell, comprises a cell penetrating amino acid sequence comprising or consisting of the sequence of:

(SEQ ID NO: 3)
AGYLLGO*INLKALAALAKAIL;
(SEQ ID NO: 4)
AGYLLGK*INLKALAALAKAIL;
(SEQ ID NO: 5)
AGYLLGDab*INLKALAALAKAIL;
(SEQ ID NO: 6)
AGYLLGDabINLKALAALAKAIL;
(SEQ ID NO: 7)
AGYLLGDap*INLKALAALAKAIL; or
(SEQ ID NO: 8)
AGYLLGO*INLKALAALAAKIL,

in particular:

(SEQ ID NO: 3)
AGYLLGO*INLKALAALAKAIL;
(SEQ ID NO: 4)
AGYLLGK*INLKALAALAKAIL;
(SEQ ID NO: 5)
AGYLLGDab*INLKALAALAKAIL;
or
(SEQ ID NO: 8)
AGYLLGO*INLKALAALAAKIL,

wherein * indicates that the peptide continues from the side chain amino group and not from the α-amino group.

In another embodiment, membrane-permeable construct for transport of cargo across a lipid membrane and subsequent delivery of said cargo into a cell, comprises a cell penetrating amino acid sequence comprising or consisting of the sequence of:

(SEQ ID NO: 9)
AGYLLGO*LKALAALAKAIL;
(SEQ ID NO: 10)
AGYLLGDab*LKALAALAKAIL;
or
(SEQ ID NO: 11)
AGYLLGDab*LKALAALAAKIL,

in particular:

(SEQ ID NO: 10)
AGYLLGDab*LKALAALAKAIL,

wherein * indicates that the peptide continues from the side chain amino group and not from the α-amino group,

In a further embodiment, the cell penetrating amino acid sequence is optionally chemically modified at the C-terminus. Such C-terminal modifications include amidation, such as wherein the C-terminus is CONH₂. Thus, in a particular embodiment the C-terminus of the cell penetrating amino acid sequence is CONH₂.

The cell penetrating amino acid sequence may be further modified to add specific targeting elements, such as for targeting the membrane-permeable constructs described herein to specific cells or organ(s). In certain embodiments, the targeting element may be for a surface receptor or ligand present on cells of the spleen. In a particular embodiment, the targeting element is for a surface receptor or ligand on dendritic cells. Targeting elements include and may therefore be selected from any one or more of the following: peptides and/or proteins (including antibodies and fragments thereof, such as antigen binding fragments (Fabs) and heavy chain only/VHH fragments) and aptamers.

### Fatty Acids

A fatty acid is a carboxylic acid with a long aliphatic chain which is either saturated or unsaturated. Short chain fatty acids are fatty acids with aliphatic tails of five or fewer carbon atoms. Medium chain fatty acids are fatty acids with aliphatic tails of six to twelve carbon atoms. Long chain fatty acids are fatty acids with aliphatic tails of 13 to 21 carbon atoms. Very long chain fatty acids are fatty acids with aliphatic tails of 22 carbon atoms or more.

The fatty acid chain of the present invention may be a saturated carbon chain or an unsaturated carbon chain, but preferably is a saturated carbon chain. Saturated fatty acids have no carbon to carbon double bonds. Unsaturated fatty acids have one or more (e.g. one to four such as one or two) carbon to carbon double bonds. The carbon to carbon double bonds can give either cis or trans isomers.

A cis configuration means that two hydrogen atoms adjacent to the double bond protrude out on the same side of the chain. The rigidity of the double bond freezes its conformation and in the case of the cis isomer, causes the chain to bend and restricts the conformational freedom of the fatty acid. The more double bonds the chain has in the cis configuration, the less flexibility it has.

A trans configuration means that the adjacent two hydrogen atoms lie on opposite sides of the chain. Consequently, they do not cause the chain to bend much, and their shape is similar to straight saturated fatty acids.

A saturated fatty acid chain having 16 carbon atoms is also referred to as palmitic acid. A saturated fatty acid chain having 18 carbon atoms is also referred to as stearic acid. A saturated fatty acid chain having 20 carbon atoms is also referred to as arachidic acid. A saturated fatty acid chain having 22 carbon atoms is also referred to as behenic acid.

An example of an unsaturated fatty acid having 16 carbon atoms is palmitoleic acid or sapienic acid. An example of an unsaturated fatty acid having 18 carbons atoms is oleic acid or elaidic acid. An example of an unsaturated fatty acid having 20 carbon atoms is arachidonic acid or eicosapentaenoic acid. An example of an unsaturated fatty acid having 22 carbon atoms is erucic acid or docosahexaenoic acid.

The cell penetrating amino acid sequences described herein have fatty acid chains attached thereto. The fatty acid chain may also be referred to as a fatty acid moiety. The fatty acid chain is attached to the amino acid sequence or peptide by a covalent bond. The fatty acid chain may be attached to the peptide via a linker molecule. In a preferred embodiment of the invention, the fatty acid is attached to the N-terminus of the cell penetrating amino acid sequence. In a still preferred embodiment, the fatty acid is attached to the N-terminal amine of the amino acid at position 1, such as position 1 of SEQ ID NO: 1 or SEQ ID NO: 2.

A linker may separate the peptide and the fatty acid chain. The linker may be a chemical moiety that contains two reactive groups/functional groups, one of which can react with the peptide and the other with the fatty acid chain. The two reactive/functional groups of the linker are linked via a linking moiety or spacer wherein the linking moiety or spacer does not interfere with the coupling of the linker to the peptide and the fatty acid chain. The linker can be made up of amino acids linked together by peptide bonds.

The fatty acid chain may have 16 to 22 carbon atoms, 16 to 20 carbon atoms, 16 to 18 carbon atoms, 16 carbon atoms, 18 to 22 carbon atoms, 18 to 20 carbon atoms, 18 carbon atoms, 20 to 22 carbon atoms, 20 carbon atoms or 22 carbon atoms. The fatty acid chain preferably has 18 to 22 carbon atoms. Alternatively or in addition, the fatty acid chain has 16, 18, 20 or 22 carbon atoms. In a certain embodiment, the fatty acid chain has 16, 18 or 20 carbon atoms. In a particularly preferred embodiment, the fatty acid chain has 18 carbon atoms.

Thus, in some embodiments the membrane-permeable construct for use in the transport of mRNA across a lipid membrane and subsequent delivery of said mRNA into a cell as described herein, comprises or consists of:
C16-SEQ ID NO: 3) (referred to herein as NF419);
C18-(SEQ ID NO: 3) (referred to herein as NF55);
C20-(SEQ ID NO: 3) (referred to herein as NF410);
C22-(SEQ ID NO: 3) (referred to herein as NF411);
C16-SEQ ID NO: 4) (referred to herein as NF420);
C18-(SEQ ID NO: 4) (referred to herein as NF554);
C20-(SEQ ID NO: 4) (referred to herein as NF412);
C22-(SEQ ID NO: 4) (referred to herein as NF413);
C16-SEQ ID NO: 5) (referred to herein as NF422);
C18-(SEQ ID NO: 5) (referred to herein as NF553);
C22-(SEQ ID NO: 5) (referred to herein as NF423);
C18-(SEQ ID NO: 6) (referred to herein as NF559);
C18-(SEQ ID NO: 7) (referred to herein as NF550);
C18-(SEQ ID NO: 8) (referred to herein as NF54);
C18-(SEQ ID NO: 9) (referred to herein as NF430);
C20-(SEQ ID NO: 9) (referred to herein as NF437);
C16-(SEQ ID NO: 10) (referred to herein as NF425);
C18-(SEQ ID NO: 10) (referred to herein as NF424);
C20-(SEQ ID NO: 10) (referred to herein as NF426);
C18-(SEQ ID NO: 11) (referred to herein as NF436); or
C20-(SEQ ID NO: 11) (referred to herein as NF438).

In particular embodiments, the membrane-permeable construct for use in the transport of mRNA across a lipid membrane and subsequent delivery of said mRNA into a cell as described herein, comprises or consists of:
C22-(SEQ ID NO: 3) (referred to herein as NF411);
C18-(SEQ ID NO: 4) (referred to herein as NF554);
C18-(SEQ ID NO: 5) (referred to herein as NF553);
C18-(SEQ ID NO: 8) (referred to herein as NF54);
C18-(SEQ ID NO: 10) (referred to herein as NF424);
C20-(SEQ ID NO: 10) (referred to herein as NF426); or
C18-(SEQ ID NO: 11) (referred to herein as NF436).

In one embodiment, the membrane-permeable construct for use comprises or consists of a cell penetrating amino acid sequence of SEQ ID NO: 3 and a fatty acid chain having 22 carbon atoms, e.g. wherein the fatty acid is behenic acid. This membrane-permeable construct is referred to herein as NF411. As demonstrated by the data presented herein, NF411 shows equivalent delivery of mRNA to NF54 and good improvement in delivery of mRNA over NF55.

In another embodiment, the membrane-permeable construct for use comprises or consists of a cell penetrating amino acid sequence of SEQ ID NO: 4 and a fatty acid chain having 18 carbon atoms, e.g. wherein the fatty acid is stearic acid. This membrane-permeable construct is referred to herein as NF554. As demonstrated by the data presented herein, NF554 shows similar delivery of mRNA to NF54 and an improved delivery of mRNA over NF55.

In another embodiment, the membrane-permeable construct for use comprises or consists of a cell penetrating amino acid sequence of SEQ ID NO: 5 and a fatty acid chain having 18 carbon atoms, e.g. wherein the fatty acid is stearic acid. This membrane-permeable construct is referred to herein as NF553. As demonstrated by the data presented herein, NF553 shows similar delivery of mRNA to NF54 and an improved delivery of mRNA over NF55.

In another embodiment, the membrane-permeable construct for use comprises or consists of a cell penetrating amino acid sequence of SEQ ID NO: 8 and a fatty acid chain having 18 carbon atoms, e.g. wherein the fatty acid is stearic acid. This membrane-permeable construct is referred to herein as NF54. As demonstrated by the data presented herein, NF54 has significantly improved delivery of mRNA over NF55 and a good improvement in mRNA delivery in vivo over NF55.

In another embodiment, the membrane-permeable construct for use comprises or consists of a cell penetrating amino acid sequence of SEQ ID NO: 10 and a fatty acid chain having 18 carbon atoms, e.g. wherein the fatty acid is stearic acid. This membrane-permeable construct is referred to herein as NF424. As demonstrated by the data presented herein, NF424 shows a good improvement in delivery of mRNA over NF54 and significantly improved delivery of mRNA over NF55. Also as demonstrated herein, NF424 shows significantly improved delivery of mRNA in vivo over both NF54 and NF55.

In another embodiment, the membrane-permeable construct for use comprises or consists of a cell penetrating amino acid sequence of SEQ ID NO: 10 and a fatty acid chain having 20 carbon atoms, e.g. wherein the fatty acid is arachidic acid. This membrane-permeable construct is referred to herein as NF426. As demonstrated by the data presented herein, NF426 shows equivalent delivery of mRNA to NF54 and good improvement in delivery of mRNA over NF55. Also as demonstrated herein, NF426 shows a good improvement in mRNA delivery in vivo over NF55.

In another embodiment, the membrane-permeable construct for use comprises or consists of a cell penetrating amino acid sequence of SEQ ID NO: 11 and a fatty acid chain having 18 carbon atoms, e.g. wherein the fatty acid is stearic acid. This membrane-permeable construct is referred to herein as NF436. As demonstrated by the data presented herein, NF436 has significantly improved delivery of mRNA in vivo over both NF54 and NF55.

In other embodiments, the membrane-permeable construct for transport of cargo across a lipid membrane and subsequent delivery of said cargo into a cell as described herein, comprises or consists of:
C18-(SEQ ID NO: 9) (referred to herein as NF430);
C20-(SEQ ID NO: 9) (referred to herein as NF437);
C16-(SEQ ID NO: 10) (referred to herein as NF425);
C18-(SEQ ID NO: 10) (referred to herein as NF424);
C20-(SEQ ID NO: 10) (referred to herein as NF426);
C18-(SEQ ID NO: 11) (referred to herein as NF436); or
C20-(SEQ ID NO: 11) (referred to herein as NF438).

In particular further embodiments, the membrane-permeable construct for transport of cargo across a lipid membrane and subsequent delivery of said cargo into a cell as described herein, comprises or consists of:
C18-(SEQ ID NO: 10) (referred to herein as NF424);
C20-(SEQ ID NO: 10) (referred to herein as NF426); or
C18-(SEQ ID NO: 11) (referred to herein as NF436).

In one embodiment, the membrane-permeable construct comprises or consists of a cell penetrating amino acid sequence of SEQ ID NO: 10 and a fatty acid chain having 18 carbon atoms, e.g. wherein the fatty acid is stearyl. This membrane-permeable construct is referred to herein as NF424. As demonstrated by the data presented herein, NF424 shows a good improvement in delivery of cargo such as mRNA over NF54 and significantly improved delivery of over NF55. Also as demonstrated herein, NF424 shows significantly improved delivery of mRNA in vivo over both NF54 and NF55.

In another embodiment, the membrane-permeable construct comprises or consists of a cell penetrating amino acid sequence of SEQ ID NO: 10 and a fatty acid chain having 20 carbon atoms, e.g. wherein the fatty acid is arachidic acid. This membrane-permeable construct is referred to herein as NF426. As demonstrated by the data presented herein, NF426 shows equivalent delivery of cargo such as mRNA to NF54 and good improvement in delivery over NF55. Also as demonstrated herein, NF426 shows a good improvement in mRNA delivery in vivo over NF55.

In another embodiment, the membrane-permeable construct comprises or consists of a cell penetrating amino acid sequence of SEQ ID NO: 11 and a fatty acid chain having 18 carbon atoms, e.g. wherein the fatty acid is stearic acid. This membrane-permeable construct is referred to herein as NF436. As demonstrated by the data presented herein, NF436 has significantly improved delivery of mRNA in vivo over both NF54 and NF55.

### Cargo

A cargo molecule may be a substance associated with a cell penetrating peptide intended to be transported into a cell. The cargo molecule may be associated with the cell penetrating peptide either through chemical linkage via covalent bonds, or through non-covalent bond or ionic bonds or non-covalent interactions or ionic interactions. The cargo associated with the cell penetrating peptide may be transported from outside of the cell, across the membrane of the cell and enter into the cell. The cargo may then be released into the cytoplasm of the cell, directed to an intracellular organelle or presented at the intracellular or extracellular cell surface. The cargo associated with the cell penetrating peptide (forming a construct or complex or nanoparticle) may enter the cell via the endosomal pathway. The construct, complex or nanoparticle may then be encapsulated in an intra-organelle, preferably in an endosomal compartment or in a lysosome, and subsequently released into the intracellular compartment or environment.

Cargoes include peptides, proteins, non-peptidic pharmaceuticals, polysaccharides, lipids (including combinations thereof including lipoproteins and glycolipids), nucleic acids (e.g. DNA, small interfering RNA (siRNA), microRNA (miRNA), short hairpin RNA (shRNA), antisense oligonucleotides, decoy DNA, plasmid DNA), small molecule drugs, and imaging agents (e.g. fluorophores, radioactive tracers and metal chelates). When the cargo molecule is a peptide, polypeptide or protein, it may comprise one or more peptides, polypeptides or proteins linked together. The peptide may be selected from a group consisting of, but not limited to, a cell or tumour targeting peptide, an aptamer, a receptor ligand, a peptide ligand, a cytotoxic peptide, a bioactive peptide, an antibody, and a diagnostic agent. When the cargo molecule is a nucleic acid, the nucleic acid may comprise one or more nucleic acids where each one encodes one peptide or polypeptide. The cargo molecule may be a combination of a protein, a lipid and/or a polysaccharide including lipoproteins and glycolipids. The cargo may be selected from a group consisting of, but not limited to, oligonucleotides including single-stranded oligonucleotides (e.g. DNA, RNA, PNA, LNA and their analogues), double-stranded oligonucleotides (e.g. siRNA, shRNA, microRNA and decoyDNA) and cyclic DNA (e.g. plasmids, such as pDNA). In certain embodiments of the present invention, the cargo is messenger RNA (mRNA). In particular, wherein the cargo is mRNA is preferred when the membrane-permeable construct described herein is for use in the transport of said mRNA cargo across a lipid membrane and subsequent delivery of the mRNA cargo into a cell.

In one embodiment, the membrane-permeable construct for use or the membrane permeable construct described herein comprises a cargo covalently attached thereto. In the case of covalent attachment, a linker moiety may optionally be present between the cell penetrating peptide and the cargo molecule, i.e. such a linker may link or connect the cell penetrating peptide and the cargo molecule together. The covalent attachment e.g. the linker moiety may be biodegradable to facilitate release of the cargo into the intracellular compartment or environment.

In a further embodiment, a complex comprises the membrane-permeable construct for use or the membrane permeable construct described herein and a cargo non-covalently interacting therewith, for example via ionic interactions. Such cargo suitably is ionic and carries negative charges. In a certain embodiment, the complex forms a nanoparticle. The complex self-assembles into a nanoparticle. The nanoparticle of the present invention comprises the amphiphilic cell penetrating amino acid sequence and associated cargo, the nanoparticle being intact upon entry into the cell. The nanoparticle of the present invention may for example be 20 to 60 nm in diameter, preferably 30 to 40 nm in diameter. Upon entry into the cell, the nanoparticle may undergo phase transition, for example inside endosomes/lysosomes, which provide a sufficiently low pH environment, thereby releasing the associated cargo into the cell.

In a preferred embodiment, the cargo non-covalently interacts (e.g. through ionic interactions) with the construct and forms a complex. In one embodiment, an siRNA cargo non-covalently interacts (e.g. via ionic interactions) with the construct and forms a complex. In a further embodiment, the complex formed of siRNA non-covalently interacting (e.g. ionically interacting) with the construct, forms a nanoparticle. In a particular embodiment, an mRNA cargo non-covalently interacts (e.g. via ionic interactions) with the construct and forms a complex. In a further embodiment, the complex formed of mRNA non-covalently interacting (e.g. ionically interacting) with the construct, forms a nanoparticle.

In certain embodiments, the mRNA cargo encodes an antigenic protein or peptide. mRNA encoding an antigenic protein or peptide delivered as described herein is predicted to be particularly useful for raising an immune response in a subject, such as a mammalian subject (e.g. a human). Thus, the cargo may form part of an mRNA vaccine, such that the membrane-permeable construct described herein with an mRNA cargo is an mRNA vaccine. In a further embodiment, the mRNA cargo encodes an antigenic protein or peptide for raising an immune response in a subject. In certain embodiments, the antigenic protein or peptide is a surface protein of a pathogen, such as a virus surface protein. Thus, in a particular embodiment the antigenic protein or peptide is a virus surface protein. Virus surface proteins include those which make up the capsid or virus envelope, e.g. a capsid protein and/or a virus envelope protein. In a particular embodiment, the antigenic protein or peptide is a vial glycoprotein. Such glycoproteins include the spike and haemagglutinin proteins, e.g. the spike protein of a coronavirus, in particular CARS-CoV2. Thus, in one embodiment the antigenic protein or peptide is the spike protein of a coronavirus, in particular CARS-CoV2. In other embodiments, the antigenic protein or peptide is a surface protein, such as the spike protein, of influenza, respiratory syncytial virus (RSV) or tick-borne encephalitis (TBE). In further other embodiments, the antigenic protein or peptide is a bacterial surface protein. In still other embodiments, the antigenic protein or peptide is a surface protein of a parasite.

In an alternative embodiment, the antigenic protein or peptide may be an antigen associated with cancer, e.g. a cancer marker protein. Such cancer-associated antigens may be suitably identified and selected depending on the particular cancer to be targeted. Thus, in further embodiments the present invention may be for use in a method of raising an anti-cancer immune response in a subject. Thus, the subject may be suffering from cancer.

### Pharmaceutical Compositions, Uses & Therapeutic Uses/Methods

In a further aspect of the invention, there is provided the membrane-permeable construct described herein for use in the transport of cargo across a lipid membrane and subsequent delivery of said cargo into a cell. As described hereinbefore, in certain embodiments the cargo is mRNA, and may be either covalently attached to the membrane-permeable construct or may non-covalently interact therewith.

The membrane-permeable constructs of the invention may be formulated for delivery in pharmaceutical compositions. Thus, in another aspect there is provided a pharmaceutical composition comprising the membrane-permeable construct as described herein. In certain embodiments, the pharmaceutical composition further comprises one or more pharmaceutically acceptable carriers, diluents and/or excipients. In further embodiments, the pharmaceutical composition optionally further comprises one or more adjuvants.

As used herein, “pharmaceutically acceptable carrier” includes any and all solvents, dispersion media, coatings, antibacterial and antifungal agents, isotonic and absorption delaying agents, and the like that are physiologically compatible. The carrier may be suitable for parenteral (e.g. topical), oral, nasal, intravenous, intramuscular, intradermal, intracranial, intraocular, intraperitoneal, subcutaneous, spinal or other parenteral routes of administration, for example by injection or infusion. For parenteral administration, the carrier preferably comprises water and may contain buffers for pH control, stabilising agents (e.g. surfactants and amino acids) and tonicity modifying agents (e.g. salts and sugars). If the composition is intended to be provided in lyophilised form for dilution at the point of use, the formulation may contain a lyoprotectant, e.g. sugars such as trehalose. For oral administration, any of the above carriers or a solid carrier, such as mannitol, lactose, starch, magnesium stearate, sodium saccharine, talcum, cellulose, glucose, sucrose and magnesium carbonate, may be employed. Alternatively, the carrier may be suitable for non-parenteral administration, such as a topical, epidermal or mucosal route of administration. The carrier may be suitable for oral administration. Depending on the route of administration, the modulator may be coated in a material to protect the compound from the action of acids and other natural conditions that may inactivate the compound.

Thus, compositions of the invention may comprise buffers (e.g. neutral buffered saline or phosphate buffered saline), carbohydrates (e.g. glucose, mannose, sucrose or dextrans), mannitol, proteins, polypeptides or amino acids such as glycine, antioxidants, bacteriostats, chelating agents such as EDTA or glutathione, solutes that render the formulation isotonic, hypotonic or weakly hypertonic with the blood of a recipient, suspending agents, thickening agents and/or preservatives. Alternatively, compositions of the invention may be formulated as a lyophilizate.

The pharmaceutical compositions of the invention may include one or more pharmaceutically acceptable salts. A “pharmaceutically acceptable salt” refers to a salt that retains the desired biological activity of the parent compound and does not impart any undesired toxicological effects. Examples of such salts include acid addition salts and base addition salts. Such salts may be prepared from pharmaceutically acceptable non-toxic bases, including organic bases (e.g. salts of primary, secondary and tertiary amines and basic amino acids) and inorganic bases (e.g. sodium, potassium, lithium, ammonium, calcium and magnesium salts).

Pharmaceutically acceptable carriers comprise aqueous carriers or diluents. Examples of suitable aqueous carriers that may be employed in the pharmaceutical compositions of the invention include water, buffered water and saline. Examples of other carriers include ethanol, polyols (such as glycerol, propylene glycol, polyethylene glycol, and the like), and suitable mixtures thereof, vegetable oils, such as olive oil, and injectable organic esters, such as ethyl oleate. In many cases, it will be desirable to include isotonic agents, for example sugars, polyalcohols such as mannitol, sorbitol, or sodium chloride in the composition.

Therapeutic compositions typically must be sterile and stable under the conditions of manufacture and storage. The composition can be formulated as a solution, microemulsion, liposome or other ordered structure suitable to high drug concentration. Pharmaceutical compositions of the invention may comprise additional active ingredients. In therapeutic applications, compounds are administered to a subject already suffering from a disorder or condition as described above, in an amount sufficient to cure, alleviate or partially arrest the condition or one or more of its symptoms. Such therapeutic treatment may result in a decrease in severity of disease symptoms, or an increase in frequency or duration of symptom-free periods. An amount adequate to accomplish this is defined as a “therapeutically effective amount”.

In prophylactic applications, formulations are administered to a subject at risk of a disorder or condition as described herein, in an amount sufficient to prevent or reduce the subsequent effects of the condition or one or more of its symptoms. An amount adequate to accomplish this is defined as a “prophylactically effective amount”. Effective amounts for each purpose will depend on the severity of the disease or injury as well as the weight and general state of the subject.

A subject for administration may be a human or non-human animal. The term “non-human animal” includes all vertebrates, e.g. mammals and non-mammals, such as non-human primates, sheep, dogs, cats, horses, cows, chickens, amphibians, reptiles, etc. Administration to humans is typical.

A pharmaceutical composition of the present invention may be administered via one or more routes of administration using one or more of a variety of methods known in the art. As will be appreciated by the skilled person, the route and/or mode of administration will vary depending upon the desired results. Examples of routes of administration for compounds or pharmaceutical compositions of the invention include intravenous, intramuscular, intradermal, intraocular, intraperitoneal, subcutaneous, spinal or other parenteral routes of administration, for example by injection or infusion. The phrase “parenteral administration” as used herein mean modes of administration other than enteral and topical administration, usually by injection. Alternatively, the pharmaceutical composition of the invention can be administered via a non-parenteral route, such as topical, epidermal or mucosal route of administration.

A suitable dose of the pharmaceutical composition of the invention may be determined by a skilled medical practitioner. Actual dosage levels of the active ingredients in the pharmaceutical compositions of the present invention may be varied so as to obtain an amount of the active ingredient that is effective to achieve the desire therapeutic response for a particular patient, composition and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

Dosage regimens may be adjusted to provide the optimum desired response, for example a therapeutic response. For example, as single dose may be administered, several divided doses maybe administered over time or the dose may be proportionally reduced or increased as indicated by the exigencies of the therapeutic situation. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Administration may be in single or multiple doses. Multiple doses may be administered via the same or different routes and to the same or different location. Alternatively, doses can be via a sustained release formulation, in which case less frequent administration is required. Dosage and frequency may vary depending on the half-life of the pharmaceutical composition in the patient and the duration of treatment desired.

In certain preferred embodiments of the present invention, pharmaceutical compositions as described herein are provided which comprise one or more (e.g. one) cell penetrating amino acid sequence as described herein in combination with a pharmaceutically acceptable carrier.

In certain embodiments, the cell is in vivo. In a further embodiment, the cell is a cell of the spleen. In a yet further embodiment, the cell is a dendritic cell (DC). In a particular embodiment, the membrane-permeable construct for use as described herein transports cargo across a lipid membrane and subsequently delivers said cargo into a cell of the spleen in vivo, such as an in vivo DC. Thus, according to a further aspect of the invention there is provided the membrane-permeable construct as described herein or the pharmaceutical composition as described herein for use in the transport of cargo across a lipid membrane and subsequent delivery of said cargo into a cell of the spleen in vivo, such as an in vivo DC. In particular, said use is provided for the membrane-permeable constructs comprising cell penetrating amino acid sequences comprising the sequence of SEQ ID NO: 2.

In another aspect of the invention, there is provided the membrane-permeable construct as described herein or the pharmaceutical composition as described herein for use in a method of raising an immune response in a subject. In a further aspect, there is provided the membrane-permeable construct or the pharmaceutical composition for use in a method of vaccinating a subject, such as vaccinating said subject against an infectious disease.

As described hereinbefore, the membrane-permeable constructs of the present invention have particularly good ability to deliver mRNA cargo in the intracellular compartment of cells, specifically into cells of the spleen in vivo, such as DCs. Thus, it is predicted that they will find utility in the raising of an immune response in a subject, wherein the mRNA cargo encodes for an antigenic protein or peptide. This is due to the efficient delivery of such mRNA to the spleen in vivo where the largest number of antigen presenting cells (APCs), such as DCs, may be found in mammals. Thus, in certain embodiments the mRNA cargo encodes an antigenic protein or peptide for raising an immune response in a subject.

In a further aspect of the present invention, there is provided a method of raising an immune response in a subject, said method comprising administering the membrane-permeable construct or pharmaceutical composition as described herein to said subject. In another aspect, there is provided a method of vaccinating a subject, said method comprising administering the membrane-permeable construct or pharmaceutical composition as described herein to said subject. In one embodiment, administering comprises a therapeutically effective amount as described hereinbefore. In another embodiment, administering comprises a prophylactically effective amount as described hereinbefore.

In a further aspect of the present invention, there is provided a use of the membrane-permeable construct or pharmaceutical composition as described herein for the manufacture of a medicament. In a particular embodiment, the medicament is a vaccine. Thus, in another aspect there is provided a use of the membrane-permeable construct or pharmaceutical composition as described herein for the manufacture of a vaccine. In one embodiment, the vaccine is for raising an immune response is a subject. Thus, in certain embodiments the vaccine comprises an mRNA cargo encoding an antigenic protein or peptide for raising an immune response in a subject. In a yet further embodiment, the vaccine comprises a therapeutically effective amount of the membrane-permeable construct or pharmaceutical composition as described herein. In another embodiment, the vaccine comprises a prophylactically effective amount of the membrane-permeable construct or pharmaceutical composition as described herein.

In some embodiments, the use or administration of the membrane-permeable construct of pharmaceutical compositions may be therapeutic as described hereinbefore. Thus, in one embodiment the subject may be suffering from a disease or disorder and thus in need thereof of the uses in and/or methods of raising an immune response described herein. In a further embodiment, the subject is suffering from cancer. According to this embodiment the raised immune response is against a cancer antigen. In other embodiments, the use or administration may be prophylactic. Thus, in a further particular embodiment the subject may be at risk of suffering from a disease or disorder, such as an infectious disease. According to this particular embodiment, the raised immune response is against an antigen of the pathogen, such as a virus (e.g. a virus surface protein). Also according to this particular embodiment, the subject may thus require vaccination against the infectious disease (e.g. a viral disease).

Thus, in a further aspect there is provided the membrane-permeable construct or the pharmaceutical composition as described herein for use in a method of raising an immune response against a virus surface protein in a subject. In a further aspect, there is provided the membrane-permeable construct or the pharmaceutical composition as described herein for use in a method of vaccinating a subject against a virus surface protein. In another aspect, there is provided a method of raising an immune response against a virus surface protein in a subject, said method comprising administering the membrane-permeable construct or the pharmaceutical composition as described herein to said subject. In a yet further aspect, there is provided a method of vaccinating a subject against a virus surface protein, said method comprising administering the membrane-permeable construct or the pharmaceutical composition as described herein to said subject. In a particular embodiment of these aspects, the virus surface protein is a capsid and/or viral envelope protein, such as a viral glycoprotein.

### In Vitro and Ex Vivo Uses

As described hereinbefore, the present membrane-permeable constructs find particular utility in vivo. However, the membrane-permeable constructs of the second aspect of the invention in particular (i.e. comprising cell penetrating amino acid sequences comprising SEQ ID NO: 2) may also be used to transfect cells in vitro or ex vivo, in particular for stable transfection. The membrane-permeable constructs of the second aspect may be used to deliver siRNA, pDNA, mRNA, peptides and/or proteins, non-peptidic pharmaceuticals, polysaccharides, lipids, small molecule drugs and imaging agents to cells, including cells in vitro.

Such cells that may be transfected include, but are not limited to, the cell lines and types: HeLa, NIH 3T3, HEK-293, CHO-K1, U2-OS and COS-7. Several hard-to-transfect cell lines, such as Jurkat, CaCo2, a human adenocarcinoma cell lines, dendritic cells and epidermis cells, may also be transfected using the membrane-permeable constructs described herein.

Unless defined otherwise, all technical and scientific terms used herein have the meaning commonly understood by a person skilled in the art to which this invention belongs. As used herein, the term “about” when used herein includes up to and including 10% greater and up to and including 10% lower than the value specified, suitably up to and including 5% greater and up to and including 5% lower than the value specified, especially the value specified. The term “between” as used herein includes the values of the specified boundaries.

Throughout the specification and the claims which follow, unless the context requires otherwise, the word “comprise”, and variations thereof such as “comprises” and “comprising”, will be understood to imply the inclusion of a stated integer, step, group of integers or group of steps but not to the exclusion of any other integer, step, group of integers or group of steps.

In addition, as used herein and the appended claims, the singular forms “a”, “an”, and “the” include plural referents unless the content clearly dictates otherwise. Thus, for example reference to “a cell penetrating amino acid sequence” includes two or more such sequences, or reference to “an mRNA” includes two or more such mRNA molecules and the like.

It will be understood that all embodiments described herein may be applied to all aspects of the invention and vice versa, and such combinations would be readily apparent from the description provided herein and to those skilled in the art.

Other features and advantages of the present invention will be apparent from the description provided herein. It should be understood, however, that the description and the specific examples while indicating preferred embodiments of the invention are given by way of illustration only, since various changes and modifications will become apparent to those skilled in the art.

### CLAUSES

A set of clauses defining the invention, its aspects and embodiments is as follows:
1. A membrane-permeable construct for use in the transport of cargo across a lipid membrane and subsequent delivery of said cargo into a cell in vivo, wherein the construct comprises a cell penetrating amino acid sequence and fatty acid chain attached to the N-terminus of said amino acid sequence, wherein the cargo is mRNA, wherein the cell penetrating amino acid sequence comprises the sequence of:

(SEQ ID NO: 1)
AGYLLG X₁*INLKALAALA X₂ IL;
or
(SEQ ID NO: 2)
AGYLLG X₁*LKALAALA X₂ IL,

wherein X₁ represents Lys (K), Orn (O), Dab or Dap,
wherein * indicates that the peptide optionally continues from the side chain amino group and not from the α-amino group,
wherein X₂ represents KA or AK, and
wherein the cell penetrating amino acid sequence is optionally chemically modified at the C-terminus.
2. The membrane-permeable construct for use of clause 1, wherein the mRNA cargo encodes an antigenic protein or peptide for raising an immune response in a subject.
3. The membrane-permeable construct for use of clause 1 or clause 2, wherein the cell is a cell of the spleen, such as a dendritic cell (DC).
4. The membrane-permeable construct for use of any one of clauses 1 to 3, wherein the mRNA encodes an antigenic protein or peptide, such as a virus surface protein, in particular a capsid and/or viral envelope protein, such as a viral glycoprotein.
5. A membrane-permeable construct for transport of cargo across a lipid membrane and subsequent delivery of said cargo into a cell, wherein the construct comprises a cell penetrating amino acid sequence and fatty acid chain attached to the N-terminus of said amino acid sequence, wherein the cell penetrating amino acid sequence comprises the sequence of:

(SEQ ID NO: 2)
AGYLLG X₁* LKALAALA X₂ IL,

wherein X₁ represents Lys (K), Orn (O), Dab or Dap,
wherein * indicates that the peptide continues from the side chain amino group and not from the α-amino group, and
wherein X₂ represents KA or AK.
6. The membrane-permeable construct for use or the membrane-permeable construct of any one of clauses 1 to 5, wherein X₁ is K, Orn (O) or Dab, in particular Dab.
7. The membrane-permeable construct for use or the membrane-permeable construct of any one of clauses 1 to 6, wherein the peptide continues from the side chain amino group and not from the α-amino group at the * position.
8. The membrane-permeable construct for use or the membrane-permeable construct of any one of clauses 1 to 7, wherein X₂ is KA.
9. The membrane-permeable construct for use or the membrane-permeable construct of any one of clauses 1 to 7, wherein X₂ is AK.
10. The membrane-permeable construct for use or the membrane-permeable construct of any one of clauses 1 to 9, wherein the cell penetrating amino acid sequence comprises one or more further amino acid substitution at positions 9 to 21 of SEQ ID NO: 1 or at positions 9 to 19 of SEQ ID NO: 2.
11. The membrane-permeable construct for use of the membrane-permeable construct of any one of clauses 1 to 10, wherein the fatty acid chain has 16 to 22 carbons atoms, such as 18 to 22 carbon atoms.
12. The membrane-permeable construct for use or the membrane-permeable construct of clause 11, wherein the fatty acid chain has 16, 18, 20 or 22 carbon atoms, such as 18, 20 or 22 carbon atoms, in particular 18 carbon atoms.
13. The membrane-permeable construct for use or the membrane-permeable construct of any one of clauses 1 to 12, wherein the cell penetrating amino acid sequence is chemically modified at the C-terminus, such as amidated, such as wherein the C-terminus is CONH₂.
14. The membrane-permeable construct for use of any one of clauses 1 to 13, wherein the cell penetrating amino acid sequence comprises the sequence of:

(SEQ ID NO: 3)
AGYLLGO*INLKALAALAKAIL;
(SEQ ID NO: 4)
AGYLLGK*INLKALAALAKAIL;
(SEQ ID NO: 5)
AGYLLGDab*INLKALAALAKAIL;
(SEQ ID NO: 6)
AGYLLGDabINLKALAALAKAIL;
(SEQ ID NO: 7)
AGYLLGDap*INLKALAALAKAIL;
or
(SEQ ID NO: 8)
AGYLLGO*INLKALAALAAKIL,

in particular:

(SEQ ID NO: 3)
AGYLLGO*INLKALAALAKAIL;
(SEQ ID NO: 4)
AGYLLGK*INLKALAALAKAIL;
(SEQ ID NO: 5)
AGYLLGDab*INLKALAALAKAIL;
or
(SEQ ID NO: 8)
AGYLLGO*INLKALAALAAKIL,

wherein * indicates that the peptide continues from the side chain amino group and not from the α-amino group,
such as wherein the membrane-permeable construct comprises:
   C16-(SEQ ID NO: 3) (referred to herein as NF419);
   C18-(SEQ ID NO: 3) (referred to herein as NF55);
   C20-(SEQ ID NO: 3) (referred to herein as NF410);
   C22-(SEQ ID NO: 3) (referred to herein as NF411);
   C16-(SEQ ID NO: 4) (referred to herein as NF420);
   C18-(SEQ ID NO: 4) (referred to herein as NF554);
   C20-(SEQ ID NO: 4) (referred to herein as NF412);
   C22-(SEQ ID NO: 4) (referred to herein as NF413);
   C16-SEQ ID NO: 5) (referred to herein as NF422);
   C18-(SEQ ID NO: 5) (referred to herein as NF553);
   C22-(SEQ ID NO: 5) (referred to herein as NF423);
   C18-(SEQ ID NO: 6) (referred to herein as NF559);
   C18-(SEQ ID NO: 7) (referred to herein as NF550);
   C18-(SEQ ID NO: 8) (referred to herein as NF54);
   C18-(SEQ ID NO: 9) (referred to herein as NF430);
   C20-(SEQ ID NO: 9) (referred to herein as NF437);
   C16-(SEQ ID NO: 10) (referred to herein as NF425);
   C18-(SEQ ID NO: 10) (referred to herein as NF424);
   C20-(SEQ ID NO: 10) (referred to herein as NF426);
   C18-(SEQ ID NO: 11) (referred to herein as NF436); or
   C20-(SEQ ID NO: 11) (referred to herein as NF438),
in particular:
   C22-(SEQ ID NO: 3) (referred to herein as NF411);
   C18-(SEQ ID NO: 4) (referred to herein as NF554);
   C18-(SEQ ID NO: 5) (referred to herein as NF553);
   C18-(SEQ ID NO: 8) (referred to herein as NF54);
   C18-(SEQ ID NO: 10) (referred to herein as NF424);
   C20-(SEQ ID NO: 10) (referred to herein as NF426); or
   C18-(SEQ ID NO: 11) (referred to herein as NF436).
15. The membrane-permeable construct of any one of clauses 1 to 13, wherein the cell penetrating amino acid sequence comprises the sequence of:

(SEQ ID NO: 9)
AGYLLGO*LKALAALAKAIL;
(SEQ ID NO: 10)
AGYLLGDab*LKALAALAKAIL;
or
(SEQ ID NO: 11)
AGYLLGDab*LKALAALAAKIL,

in particular:

(SEQ ID NO: 10)
AGYLLGDab*LKALAALAKAIL,

wherein * indicates that the peptide continues from the side chain amino group and not from the α-amino group,
such as wherein the membrane-permeable construct comprises:
   C18-(SEQ ID NO: 9) (referred to herein as NF430);
   C20-(SEQ ID NO: 9) (referred to herein as NF437);
   C16-(SEQ ID NO: 10) (referred to herein as NF425);
   C18-(SEQ ID NO: 10) (referred to herein as NF424);
   C20-(SEQ ID NO: 10) (referred to herein as NF426);
   C18-(SEQ ID NO: 11) (referred to herein as NF436); or
   C20-(SEQ ID NO: 11) (referred to herein as NF438),
in particular:
   C18-(SEQ ID NO: 10) (referred to herein as NF424);
   C20 (SEQ ID NO: 10) (referred to herein as NF426); or
   C18-(SEQ ID NO: 11) (referred to herein as NF436).
16. The membrane-permeable construct of any one of clauses 5 to 15 for use in the transport of cargo across a lipid membrane and subsequent delivery of said cargo into a cell.
17. The membrane-permeable construct for use or the membrane-permeable construct of any one of clauses 1 to 16, wherein the construct further comprises a cargo covalently attached thereto.
18. A complex comprising a membrane-permeable construct for use or the membrane-permeable construct of any one of clauses 1 to 16 and a cargo non-covalently interacting therewith, such as via ionic interactions.
19. The membrane-permeable construct for use, the membrane-permeable construct or the complex of any one of clauses 16 to 18, wherein the cargo is mRNA.
20. The membrane-permeable construct for use, the membrane-permeable construct or the complex of any one of clauses 16 to 19, wherein the cell is in vivo, and/or wherein the cell is a cell of the spleen, such as a dendritic cell (DC).
21. The membrane-permeable construct for use, the membrane-permeable construct or the complex of clause 19 or clause 20, wherein the mRNA encodes an antigenic protein or peptide, such as a virus surface protein, in particular a capsid and/or viral envelope protein, such as a viral glycoprotein.
22. A pharmaceutical composition comprising the membrane-permeable construct or the complex of any one of clauses 5 to 21 and a pharmaceutically acceptable carrier, optionally further comprising one or more adjuvants.
23. The membrane-permeable construct for use, the membrane-permeable construct or the complex of any one of clauses 1 to 21 or the pharmaceutical composition of clause 22 for use in a method of raising an immune response in a subject.

The invention will now be described using the following, non-limiting examples:

### EXAMPLES

### Example 1: Materials and Methods

### Transfection Reagents

Cell-penetrating peptides were synthesised on an automated peptide synthesiser (Biotage Initiator+ Alstra) using the fluorenylmethyloxycarbonyl (Fmoc) solid phase peptide synthesis strategy with Rink-amide ChemMatrix resin (0.41 mmol g-1 loading) to obtain C-terminally amidated peptides. The fatty acid was coupled manually to the N-terminus of the peptide overnight, at room temperature with 5 eq. of fatty acid. For the synthesis of “kinked” peptides the Boc-L-Orn(Fmoc)-OH, Boc-L-Lys(Fmoc)-OH, Boc-L-Dab(Fmoc)-OH, Boc-L-Dap(Fmoc)-OH (Iris Biotech, Germany) was used to continue the synthesis from the side-chain amino group. The reaction was carried out in DMF using HOBT/HBTU for manual or DIC/Oxyma for machine synthesis as coupling reagents, with DIEA as an activator base. The cleavage was performed with trifluoroacetic acid, 2.5% triisopropylsilane and 2.5% water for 2 hours at room temperature. Peptides were purified by reversed-phase high-performance liquid chromatography on a C4 column (Phenomenex Jupiter C4, 5 μm, 300 Å, 250×10 mm) using a gradient of acetonitrile/water containing 0.1% TFA. The molecular weight of the peptides was analysed by matrix-assisted laser desorption-ionization/time of flight mass spectrometry (Brucker Microflex LT/SH, USA). The concentration of the peptides was determined based on dilutions of accurately weighed substances and absorption of tyrosine, where applicable.

### In Vitro Methods:

### Cell Culture Maintenance

Adherent CHO-K1 cells were grown in Dulbecco's Modified Eagle's Medium (DMEM), which were supplemented with 0.1 mM non-essential amino acids, 1.0 mM sodium pyruvate, 100 U/ml penicillin, and 100 mg/ml streptomycin. For complete media, 10% (final) fetal bovine serum (FBS) was added. Cells were maintained in a humidified incubator at 37 °C, 5% CO2. Cell viability and confluence was assessed daily and the cell density was reduced regularly. For cell counting, we used the CytoSMART cell counter accompanied by 0.4% trypan blue staining prior measurement. The multi-well plates with cell cultures used for experiments were incubated in a humidified incubator at 37 °C, 5% CO₂.

### Transfection Complex Preparation

For in vitro experiments 0.1 μg of nucleic acid (plasmid DNA (pDNA) or mRNA) was used per 96-well plate well. For CPP/NA complex formation, the diluted NA was mixed with CPP in water. Complexes were formed based on the theoretical charge ratio (CR) of positive charges from the peptide in excess to the negative charges from nucleic acid backbone, at charge ratio 3:1 with peptide in excess. Commercial reagent Lipofectamine3000 (LF3000) was used according to the manufacturer's recommendations for the transfection of adherent cells.

### Reporter Quantitation for In Vitro Experiments

For reporter luminescence (expressed from firefly luciferase encoding mRNA or plasmid pDNA) assessment in adherent cell culture, 10,000 cells per 96-well plate well were seeded in 100 μl of media one day prior to transfection. Shortly before transfection, cell media was replaced with 100 μl of serum containing (10% foetal bovine serum) DMEM media. CPPs and mRNA or pDNA were mixed at a charge ratio 3:1 in ultrapure water and incubated at room temperature before addition to cells in serum containing media. Cells were transfected with 0.1 μg of mLuc per well.

24 hours post-transfection, the media was aspirated, cells were washed with 1×PBS, and 30 μl of lysis buffer (0.1% Triton X100 in 1×PBS) was added. Cells were incubated with lysis buffer for 20 min. to lyse the cells. From the lysate, 20 μl was transferred to a black frame white well 96-well plate for luminescence measurement after addition of substrate luciferin (PerkinElmer) in buffer. The luminescence signal was detected with GloMax® 96 microplate luminometer equipped with GloMax® 1.9.2 software (Promega). RLU values were converted to RLU/mg by normalization to total protein in cell lysate, reflecting total number of cells. For protein detection, a Pierce™ BCA Protein Assay Kit was used.

### In Vivo Methods:

### Transfection Complex Preparation

The transfection complexes (CPP/mRNA) were formed using 10 μg of mRNA mixed with each peptide at indicated charge ratios (CRs) in MQ water in a final volume of 100 μl. CRs were calculated theoretically and account for both the positive charges of the peptide and negative charges of the nucleic acid. The transfection complexes (CPP/mRNA and CPP/pDNA) were formed using 50 μg of mRNA mixed with each peptide at indicated charge ratios (CRs) in MQ water in a final volume of 168 μl. The complexes were incubated for 30 minutes at room temperature and then mixed with equal volume of 10% glucose (resulting in a final solution of 5% glucose in a total volume of 200 μl for 10 μg dose groups and 336 μl for 50 μg dose groups) and immediately injected intravenously via the tail vein.

Reporter Quantification from the Tissue Homogenate

The reporter gene (luciferase) expression levels were evaluated from the tissue homogenate post mortem 16-24 h after single injection.

Reporter Quantification with the Whole Body Imager

100 μl of luciferin (PerkinElmer), at a concentration of 15 mg/ml, was intraperitoneally injected into the mice. After 10 min, the mice were imaged using the In Vivo Imaging System (IVIS Lumina II, PerkinElmer).

### Immunophenotyping the Spleen Cell Subtypes

To analyse the cell subtypes that were transfected with the CPP, 30 μg of luciferase-encoding mRNA was complexed with the CPP at CR2 in water and injected i.v. 16 h post-injection, the spleens were collected into ice-cold dissociation buffer (1×PBS without Ca²⁺ and Mg²⁺ supplemented with 2 mM EDTA). Following gentle mechanical dissociation by grinding, splenic cells were strained with 100 μm cell strainer and centrifuged for 5 min at +4 °C, 300×g. A 2-minute incubation with 1 ml of RBC lysis buffer ACK (Thermo) was used for the red blood cell lysis. Flow cytometry buffer (dissociation buffer supplemented with 0.5% (w/v) BSA) was added, cells were centrifuged for 5 min at +4 °C, 300×g and counted. 20 million cells were 10 incubated with Truestain FcX anti-mouse CD16/32 (Biolegend) and stained with antibodies and markers listed in Table 1. Cells were washed three times and sorted using SONY MA900 sorter with a 100 μm sorting chip. The cell subtypes were defined as follows: CD4+/CD8+=T cells; Cd11c+=DC cells; Cd11b+/F4+=macrophages.

TABLE 1
Reagents and Antibodies used for Flow Cytometry

| | | | Dilution | |
|---|---|---|---|---|
| | | | used for | |
| Antigen | Clone | Fluorophore | staining | Source |
| Fixable | | eFluor ™ 506 | 1:800 | Thermo Fisher |
| Viability | | | | Scientific |
| Dye | | | | |
| Cd45 | 30-F11 | AlexaFluor700 | 1:400 | Biolegend |
| Cd11b | M1/70 | PE-Dazzle | 1:800 | Biolegend |
| Cd11c | N418 | APC-Cy7 | 1:400 | Biolegend |
| Cd19 | 1D3/CD19 | PerCP-Cy5.5 | 1:200 | Biolegend |
| Cd8 | 53-5.8 | BrilliantViolet650 | 1:200 | Biolegend |
| Cd4 | RM4-5 | BrilliantViolet570 | 1:400 | Biolegend |
| F4/80 | BM8 | PE-Cy7 | 1:400 | Biolegend |

### Example 2: Delivery of Reporter-Expressing mRNA with Cell Penetrating Peptides (CPPs) in Cell Culture

The present inventors have previously reported CPPs developed from the NickFect (NF) and PepFect (PF) families for plasmid (pDNA), siRNA and miRNA delivery (see, e.g. WO 2020/144317, WO 2010/039088 and WO 2012/113846). With the emergence of mRNA-based therapeutics, it was decided to investigate peptides for their ability to deliver mRNA. It was assumed that CPPs that are efficient for siRNA and miRNA delivery, e.g. histidine containing CPPs (Porosk et. al. (2019)), may be good candidates for the delivery of mRNA. Therefore, CPPs from each main family (NF55-, PF14- and NF70-derived CPPs) were tested for mRNA delivery/transfection. Naked mRNA treated and untreated cells were used as control groups. LF3000 was used to compare the transfection efficacy of liposome-based transfection reagents with the CPPs (FIG. 1).

As can be seen from the data in FIG. 1 and Table 2, in contrast to the above mentioned assumption that CPPs which are efficient for siRNA and miRNA delivery would also be good for mRNA, the delivery of mRNA follows a different logic than the delivery of other nucleic acids, such as pDNA, siRNA and miRNA. Therefore, surprisingly if a CPP is an efficient transfection vector for siRNA, miRNA or pDNA delivery, it cannot be assumed that it will be efficient as well for the delivery of mRNA. In particular, PF14 and NF1 are known to be efficient delivery vectors for pDNA, but the results herein demonstrate that they are not efficient for mRNA transfection. Moreover, NF71 and NF70 that are very efficient for siRNA and miRNA delivery are not efficient for mRNA delivery.

In comparison to PF14, NF1, NF71 and NF70, the inventors found that other CPPs were much more efficient for the delivery of mRNA. Out of all the tested peptides, NF424, NF411, NF426, NF553, NF554, NF410, NF54 and NF55 were the most efficient (see FIG. 1). Several other new peptides also showed very promising efficacies as mRNA delivery vectors.

These experiments revealed that the most efficient CPPs were NF55-based, not histidine-rich (such as NF70 and NF71) nor PF14-based. Therefore, NF55-based peptides were investigated and evaluated further, especially in vivo.

TABLE 2
Delivery of Reporter mRNA with CPPs in Cell Culture -
Evaluation of Reporter Levels Post-Transfection

| Peptide | Average RLU/mg |
|---|---|
| NF424 | 2 590 000 000 |
| NF54 | 2 160 000 000 |
| NF411 | 2 090 000 000 |
| NF426 | 1 960 000 000 |
| NF553 | 1 750 000 000 |
| NF554 | 1 690 000 000 |
| NF55 | 1 270 000 000 |
| NF410 | 1 080 000 000 |
| NF430 | 766 000 000 |
| NF413 | 678 000 000 |
| NF559 | 563 000 000 |
| NF420 | 548 000 000 |
| NF423 | 556 000 000 |
| NF419 | 491 000 000 |
| NF550 | 441 000 000 |
| NF436 | 380 000 000 |
| NF412 | 393 000 000 |
| NF422 | 159 000 000 |
| NF425 | 106 000 000 |
| PF14 | 87 500 342 |
| PF1452 | 49 508 090 |
| NF1 | 39 779 660 |
| NF71 | 3 505 987 |
| Lipofectamine3000 | 2 889 184 |
| NF70 | 919 720 |
| NF72 | 78 023 |
| mRNA (naked) | 4 332 |

### Example 3: Comparison of Reporter Levels in Cells Post-Transfection of pDNA or mRNA Delivered by CPPs

CPPs from PepFect, NickFect, and histidine-rich families were tested in parallel for pDNA and mRNA transfection (FIG. 2).

The results demonstrate that PF14 and NF71, which are very efficient transfection reagents for pDNA delivery, were not effective tools for mRNA transfection/delivery. This is surprising as both mRNA and pDNA are nucleic acid polymers and in these experiments encoded the same reporter (firefly luciferase). In contrast, NF55 was very efficient for mRNA transfection, but not for pDNA transfection (see FIG. 2).

As previously highlighted, NF71 is very efficient for siRNA and miRNA transfections. However, surprisingly NF71 is not an efficient delivery vector for mRNA, although siRNA, miRNA, and mRNA are all RNA types of cargo and exert their activity in the same cellular compartment (the cytoplasm).

These results show that the ability of a CPP to deliver mRNA cannot be predicted by the corresponding efficacy of transfecting pDNA, and that specific CPPs are required to deliver mRNA.

### Example 4: Biodistribution of Reporter Levels Post-Transfection of mRNA or pDNA with NFs or PFs In Vivo

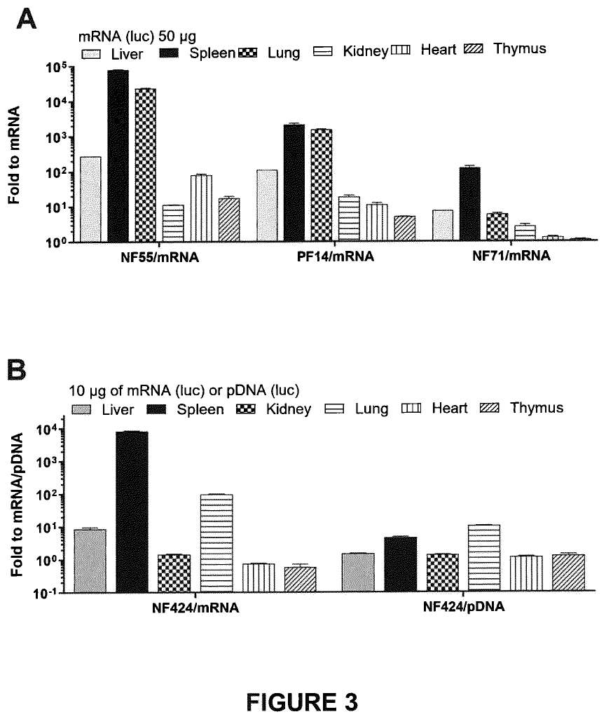

In order to determine to which organs in vivo mRNAs may be delivered to with NickFect or PepFect CPPs, mRNA complexes with NF55, NF71 and PF14 were formed and injected through the tail vein of Balb/c mice (FIG. 3A). The organs were harvested 24 hours after the single injection and reporter signal was measured from whole tissue homogenates. The biodistribution of reporter levels was measured in liver, spleen, lung, kidney, heart and thymus.

Interestingly, with NF55, delivery of mRNA to the spleen was 3.5 times higher than delivery to the lungs and much higher than in any other organ (see FIG. 3A and Table 3). At the same time, with PF14, there was no significant difference in the measured reporter level in the spleen compared to lung tissue. This data demonstrates the selectivity of NF55 to transfect mRNA into the spleen in vivo. NF71 also delivered mRNA mostly into the spleen, with very low transfection levels seen in other organs.

In order to compare the biodistribution profiles between harvested organs when using either mRNA or pDNA as cargo, NF424 was used to transfect these 2 types of nucleic acids in vivo (FIG. 3B and Table 4). These data show that, when pDNA was transfected, the main expression was found in lungs and liver. However, for mRNA delivery, the highest expression levels were found in the spleen of treated mice (i.e. the organ of interest).

TABLE 3
Biodistribution of Reporter Levels Post-Transfection
of mRNA or pDNA with NFs or PFs CPPs In
Vivo - NF55, PF14 and NF71 Delivery of mRNA

| Peptide | Spleen | Lung | Liver |
|---|---|---|---|
| NF55 | 79 879 | 23 465 | 284 |
| PF14 | 2 159 | 1 565 | 112 |
| NF71 | 124 | 6 | 8 |

TABLE 4
Biodistribution of Reporter Levels Post-Transfection
of mRNA or pDNA with NFs or PFs CPPs In
Vivo - NF424 Delivery of mRNA and pDNA

| | Spleen | Lung | Liver |
|---|---|---|---|
| Fold/mRNA | 8 330 | 98 | 9 |
| Fold/pDNA | 5 | 11 | 2 |

### Example 5: Live Animal Imaging 1-48 Hours Post-Administration of NF424/mRNA or NF424/pDNA Complexes

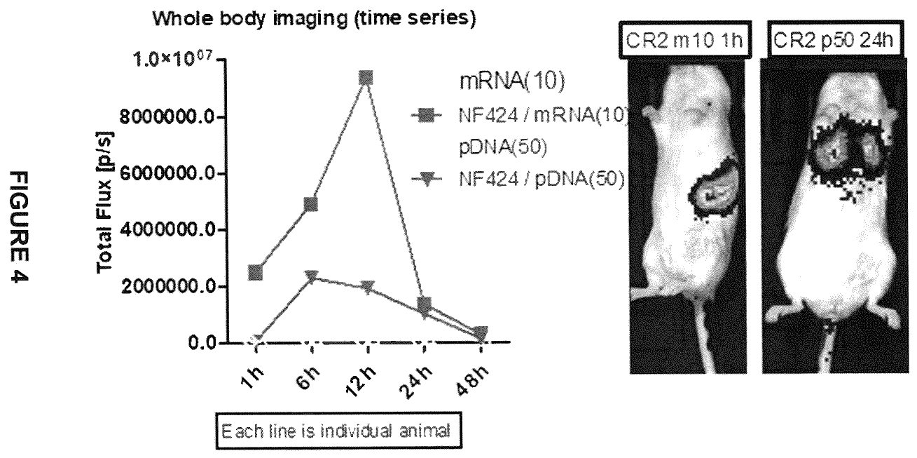

Live animal imaging was used to track the timepoints of mRNA and pDNA transfection with NFs (FIG. 4). The CPP used was NF424, complexes were formed in parallel with mRNA or pDNA and injected through the tail vein of BALB/c mice. The reporter gene expression levels were evaluated with full body imager at 1 h, 6 h, 12 h, 24 h and 48 h post-injection.

It should be noted that the spleen contains the largest number of antigen-presenting cells (APCs) in the mammalian organism (see hereinbefore). That is why it is an attractive target for immunisation approaches.

As can be seen in FIG. 4 (left panel), after 1 hour it was already possible to detect the reporter signal in the spleens of mice injected with NF424/mRNA complexes. For pDNA, the reporter signal was visible in the lungs only 6 hours after the injection. Despite the injected dose of pDNA being 5 times higher (50 μg) than the dose of mRNA (10 μg), the detected signal was much higher from mRNA. This data shows that NF424 delivers mRNA mostly to the spleen and pDNA to the lungs, but is much more effective at delivering mRNA than pDNA in vivo.

### Example 6: In Vivo Delivery of mRNA with CPPs in the Spleen

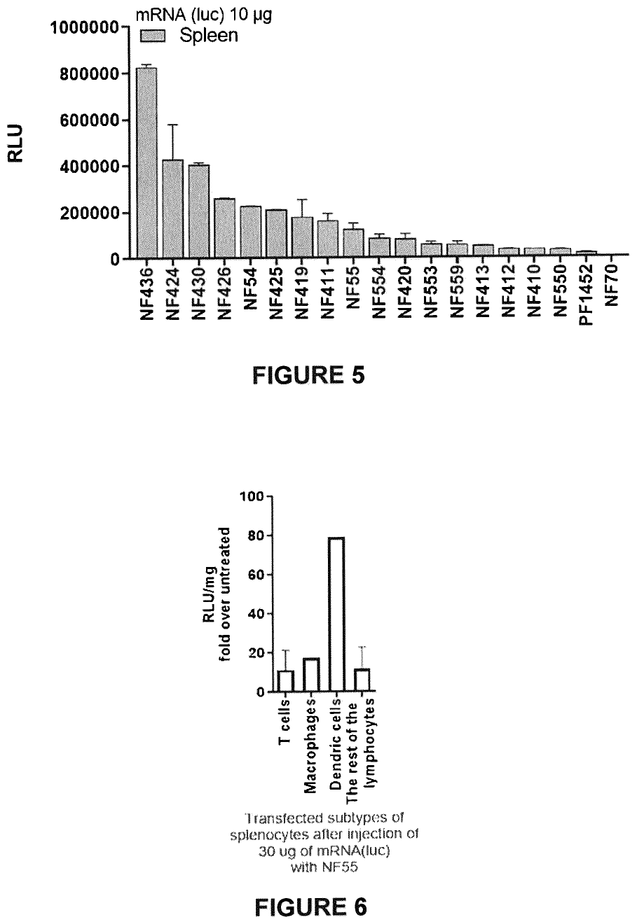

Expanding on the in vitro results of Example 2, the CPPs which were the most efficient for mRNA delivery were tested in vivo. CPPs and mRNA complexes were injected through the tail vein of BALB/c mice, spleens were harvested 16 hours after the single injection and reporter signal was measured from the whole tissue homogenate (FIG. 5 and Table 5).

The highest reporter levels after mRNA delivery into the spleen in vivo were achieved with NF436, NF424 and NF430. However, other vectors such as NF426, NF54, NF425, NF419, NF411 and NF55 also delivered mRNA into the spleen in vivo with high efficacy. The best CPP, NF436, showed 43-times more efficient mRNA in vivo spleen delivery than PF1452 and 100-times more efficient than NF70 (see FIG. 5 and Table 5).

Therefore, while many CPPs are able to transfect nucleic acids, only few are able to efficiently deliver mRNAs into the spleen in vivo. The results presented herein have demonstrated several CPPs suitable for mRNA delivery, such as delivery to the spleen in vivo, and that it is important to choose the appropriate CPP for mRNA in vivo delivery.

TABLE 5
Delivery of mRNA with CPPs In
Vivo - Reporter Levels in the Spleen

| Peptide | RLU |
|---|---|
| NF436 | 825 783 |
| NF424 | 427 566 |
| NF430 | 405 318 |
| NF426 | 259 807 |
| NF54 | 225 675 |
| NF425 | 209 700 |
| NF419 | 179 114 |
| NF411 | 161 716 |
| NF55 | 124 363 |
| NF554 | 83 650 |
| NF420 | 81 271 |
| NF553 | 57 989 |
| NF559 | 56 280 |
| NF413 | 50 495 |
| NF412 | 36 759 |
| NF410 | 36 670 |
| NF550 | 33 032 |
| PF1452 | 19 352 |
| NF70 | 8 000 |

### Example 7: Immunophenotyping the Spleen Cell Subtypes Post-Transfection of mRNA with NF55 In Vivo

In order to determine to which types of spleen cells mRNA were transfected in vivo, cells collected from the spleens of transfected animals were sorted according to their subtype and the reporter expression measured. mRNA encoding luciferase (mRNA (luc)) was used as a reporter model. It was observed that the majority of the reporter signal arose from the dendritic cells (DCs) sorted from the spleens of transfected animals (FIG. 6). This data demonstrates the selectivity of NF55 to transfect mRNA into the antigen-presenting cells of the spleen in vivo.

SEQUENCE LISTING

| Sequence Identifier | CPP Amino Acid Sequence | Comprised In |
|---|---|---|
| SEQ ID NO: 1 | AGYLLG X₁* INLKALAALA X₂ IL | NF419, NF55, NF410, |
| | | NF411, NF420, NF554, |
| | | NF412, NF413, NF422, |
| | | NF553, NF423, NF559, |
| | | NF550, NF54 |
| SEQ ID NO: 2 | AGYLLG X₁* LKALAALA X₂ IL | NF430, NF437, NF425, |
| | | NF424, NF426, NF436, |
| | | NF438 |
| SEQ ID NO: 3 | AGYLLGO*INLKALAALAKAIL | NF419, NF55, NF410, |
| | | NF411 |
| SEQ ID NO: 4 | AGYLLGK*INLKALAALAKAIL | NF420, NF554, NF412, |
| | | NF413 |
| SEQ ID NO: 5 | AGYLLGDab*INLKALAALAKAIL | NF422, NF553, NF423 |
| SEQ ID NO: 6 | AGYLLGDabINLKALAALAKAIL | NF559 |
| SEQ ID NO: 7 | AGYLLGDap*INLKALAALAKAIL | NF550 |
| SEQ ID NO: 8 | AGYLLGO*INLKALAALAAKIL | NF54 |
| SEQ ID NO: 9 | AGYLLGO*LKALAALAKAIL | NF430, NF437 |
| SEQ ID NO: 10 | AGYLLGDab*LKALAALAKAIL | NF425, NF424, NF426 |
| SEQ ID NO: 11 | AGYLLGDab*LKALAALAAKIL | NF436, NF438 |
| SEQ ID NO: 12 | AGY(PO3)LLGKTNLKALAALAKKIL | NF1 |
| SEQ ID NO: 13 | HHHHYHHGO*ILLKALKALAKAIL | NF70 |
| SEQ ID NO: 14 | HHYHHGO*ILLKALKALAKAIL | NF71 |
| SEQ ID NO: 15 | HHHHHHYHHGO*ILLKALKALAKAIL | NF72 |
| SEQ ID NO: 16 | AGYLLGKLLOOLAAAALOOLL | PF14 |
| SEQ ID NO: 17 | AGYLLGKLLOOLAOOALOOLL | PF1452 |

X₁ represents K, Orn (0), Dab or Dap; * indicates that the peptide optionally continues (SEQ ID NOs: 1 and 2) or continues from the side chain amino group and not from the α-amino group; X₂ represents KA or AK; Dab refers to 2,4-diaminobutanoic acid; and Dap refers to 2,3-diaminopropionic acid.

1. A membrane-permeable construct for use in the transport of cargo across a lipid membrane and subsequent delivery of said cargo into a cell in vivo, wherein the construct comprises a cell penetrating amino acid sequence and fatty acid chain attached to the N-terminus of said amino acid sequence, wherein the cargo is mRNA, wherein the cell penetrating amino acid sequence comprises the sequence of:
(SEQ ID NO: 1)
AGYLLG X₁* INLKALAALA X₂ IL;
or
(SEQ ID NO: 2)
AGYLLG X₁* LKALAALA X₂ IL,
wherein X₁ represents Lys (K), Orn (O), Dab or Dap,
wherein * indicates that the peptide optionally continues from the side chain amino group and not from the α-amino group,
wherein X₂ represents KA or AK, and
wherein the cell penetrating amino acid sequence is optionally chemically modified at the C-terminus.

2. The membrane-permeable construct for use of claim 1, wherein the mRNA cargo encodes an antigenic protein or peptide for raising an immune response in a subject, and/or
wherein the cell is a cell of the spleen, such as a dendritic cell (DC).

3. The membrane-permeable construct for use of claim 1, wherein the mRNA encodes an antigenic protein or peptide, such as a virus surface protein, in particular a capsid and/or viral envelope protein, such as a viral glycoprotein.

4. A membrane-permeable construct for transport of cargo across a lipid membrane and subsequent delivery of said cargo into a cell, wherein the construct comprises a cell penetrating amino acid sequence and fatty acid chain attached to the N-terminus of said amino acid sequence, wherein the cell penetrating amino acid sequence comprises the sequence of:
AGYLLG X₁*LKALAALA X₂ IL (SEQ ID NO: 2),
wherein X₁ represents Lys (K), Orn (O), Dab or Dap,
wherein * indicates that the peptide continues from the side chain amino group and not from the α-amino group, and
wherein X₂ represents KA or AK.

5. The membrane-permeable construct for use or the membrane-permeable construct of claim 1, wherein X₁ is K, Orn (O) or Dab, in particular Dab.

6. The membrane-permeable construct for use or the membrane-permeable construct of claim 1, wherein the peptide continues from the side chain amino group and not from the α-amino group at the * position.

7. The membrane-permeable construct for use or the membrane-permeable construct of claim 1, wherein X₂ is KA or X₂ is AK.

8. The membrane-permeable construct for use or the membrane-permeable construct of claim 1, wherein the cell penetrating amino acid sequence comprises one or more further amino acid substitution at positions 9 to 21 of SEQ ID NO: 1 or at positions 9 to 19 of SEQ ID NO: 2.

9. The membrane-permeable construct for use or the membrane-permeable construct of claim 1, wherein the fatty acid chain has 16 to 22 carbons atoms, such as 18 to 22 carbon atoms, and
optionally wherein the fatty acid chain has 16, 18, 20 or 22 carbon atoms, such as 18, 20 or 22 carbon atoms, in particular 18 carbon atoms.

10. The membrane-permeable construct for use or the membrane-permeable construct of claim 1, wherein the cell penetrating amino acid sequence is chemically modified at the C-terminus, such as amidated, such as wherein the C-terminus is CONH₂.

11. The membrane-permeable construct for use of claim 1, wherein the cell penetrating amino acid sequence comprises the sequence of:
(SEQ ID NO: 3)
AGYLLGO*INLKALAALAKAIL;
(SEQ ID NO: 4)
AGYLLGK*INLKALAALAKAIL;
(SEQ ID NO: 5)
AGYLLGDab*INLKALAALAKAIL;
(SEQ ID NO: 6)
AGYLLGDabINLKALAALAKAIL;
(SEQ ID NO: 7)
AGYLLGDap*INLKALAALAKAIL;
or
(SEQ ID NO: 8)
AGYLLGO*INLKALAALAAKIL,
in particular:
(SEQ ID NO: 3)
AGYLLGO*INLKALAALAKAIL;
(SEQ ID NO: 4)
AGYLLGK*INLKALAALAKAIL;
(SEQ ID NO: 5)
AGYLLGDab*INLKALAALAKAIL;
or
(SEQ ID NO: 8)
AGYLLGO*INLKALAALAAKIL,
wherein * indicates that the peptide continues from the side chain amino group and not from the α-amino group,
such as wherein the membrane-permeable construct comprises:
C16-(SEQ ID NO: 3) (referred to herein as NF419);
C18-(SEQ ID NO: 3) (referred to herein as NF55);
C20-(SEQ ID NO: 3) (referred to herein as NF410);
C22-(SEQ ID NO: 3) (referred to herein as NF411);
C16-(SEQ ID NO: 4) (referred to herein as NF420);
C18-(SEQ ID NO: 4) (referred to herein as NF554);
C20-(SEQ ID NO: 4) (referred to herein as NF412);
C22-(SEQ ID NO: 4) (referred to herein as NF413);
C16-SEQ ID NO: 5) (referred to herein as NF422);
C18-(SEQ ID NO: 5) (referred to herein as NF553);
C22-(SEQ ID NO: 5) (referred to herein as NF423);
C18-(SEQ ID NO: 6) (referred to herein as NF559);
C18-(SEQ ID NO: 7) (referred to herein as NF550);
C18-(SEQ ID NO: 8) (referred to herein as NF54);
C18-(SEQ ID NO: 9) (referred to herein as NF430);
C20-(SEQ ID NO: 9) (referred to herein as NF437);
C16-(SEQ ID NO: 10) (referred to herein as NF425);
C18-(SEQ ID NO: 10) (referred to herein as NF424);
C20-(SEQ ID NO: 10) (referred to herein as NF426);
C18-(SEQ ID NO: 11) (referred to herein as NF436); or
C20-(SEQ ID NO: 11) (referred to herein as NF438),
in particular:
C22-(SEQ ID NO: 3) (referred to herein as NF411);
C18-(SEQ ID NO: 4) (referred to herein as NF554);
C18-(SEQ ID NO: 5) (referred to herein as NF553);
C18-(SEQ ID NO: 8) (referred to herein as NF54);
C18-(SEQ ID NO: 10) (referred to herein as NF424);
C20-(SEQ ID NO: 10) (referred to herein as NF426); or
C18-(SEQ ID NO: 11) (referred to herein as NF436).

12. The membrane-permeable construct of claim 1, wherein the cell penetrating amino acid sequence comprises the sequence of:
(SEQ ID NO: 9)
AGYLLGO*LKALAALAKAIL;
(SEQ ID NO: 10)
AGYLLGDab*LKALAALAKAIL;
or
(SEQ ID NO: 11)
AGYLLGDab*LKALAALAAKIL,
in particular:
(SEQ ID NO: 10)
AGYLLGDab*LKALAALAKAIL,
wherein * indicates that the peptide continues from the side chain amino group and not from the α-amino group,
such as wherein the membrane-permeable construct comprises:
C18-(SEQ ID NO: 9) (referred to herein as NF430);
C20-(SEQ ID NO: 9) (referred to herein as NF437);
C16-(SEQ ID NO: 10) (referred to herein as NF425);
C18-(SEQ ID NO: 10) (referred to herein as NF424);
C20-(SEQ ID NO: 10) (referred to herein as NF426);
C18-(SEQ ID NO: 11) (referred to herein as NF436); or
C20-(SEQ ID NO: 11) (referred to herein as NF438),
in particular:
C18-(SEQ ID NO: 10) (referred to herein as NF424);
C20 (SEQ ID NO: 10) (referred to herein as NF426); or
C18-(SEQ ID NO: 11) (referred to herein as NF436).

13. The membrane-permeable construct of claim 4 for use in the transport of cargo across a lipid membrane and subsequent delivery of said cargo into a cell.

14. The membrane-permeable construct for use or the membrane-permeable construct of claim 1, wherein the construct further comprises a cargo covalently attached thereto.

15. A complex comprising a membrane-permeable construct for use or the membrane-permeable construct of claim 1 and a cargo non-covalently interacting therewith, such as via ionic interactions.

16. The membrane-permeable construct for use, the membrane-permeable construct or the complex of claim 13, wherein the cargo is mRNA,
optionally wherein the mRNA encodes an antigenic protein or peptide, such as a virus surface protein, in particular a capsid and/or viral envelope protein, such as a viral glycoprotein.

17. The membrane-permeable construct for use, the membrane-permeable construct or the complex of claim 13, wherein the cell is in vivo, and/or wherein the cell is a cell of the spleen, such as a dendritic cell (DC).

18. A pharmaceutical composition comprising the membrane-permeable construct or the complex of claim 4 and a pharmaceutically acceptable carrier, optionally further comprising one or more adjuvants.

19. The membrane-permeable construct for use, the membrane-permeable construct or the complex of claim 1 for use in a method of raising an immune response in a subject.

20. The pharmaceutical composition of claim 18 for use in a method of raising an immune response in a subject.
